(19) United States
(12) Patent Application Publication
(10) Pub. No.: US 2025/0250219 A1
(43) Pub. Date: Aug. 7, 2025

(54) BICYCLIC KETONE COMPOUND AND PREPARATION METHOD THEREFOR, AND PREPARATION METHOD FOR BICYCLO[3.2.1]-3-OCTANE-2,4-DIONE

(71) Applicant: SHANDONG WEIFANG RAINBOW CHEMICAL CO.,LTD, Shandong (CN)

(72) Inventors: Hongquan ZHANG, Shandong (CN); Zhiqing Li, Shandong (CN); Gen Li, Shandong (CN); Yanpeng Hou, Shandong (CN); Guangli Zhao, Shandong (CN)

(73) Assignee: SHANDONG WEIFANG RAINBOW CHEMICAL CO.,LTD, Shandong (CN)

(21) Appl. No.: 19/187,954

(22) Filed: Apr. 23, 2025

Related U.S. Application Data
(63) Continuation of application No. PCT/CN2023/126283, filed on Oct. 24, 2023.

(30) Foreign Application Priority Data
Jan. 28, 2023 (CN) 202310042247.9

(51) Int. Cl. C07C 49/753

(52) U.S. Cl. CPC C07C 49/753

(57) ABSTRACT

The present application discloses a bicyclic ketone compound and a preparation method therefor, and a preparation method for bicyclo[3.2.1]-3-octane-2,4-dione, belonging to the technical field of medicaments. 3-chlorobicyclo[3.2.1]-3-octen-2-one reacts with an alcohol in the presence of a strong alkali and a catalyst under an anhydrous condition to generate 4-alkoxybicyclo[3.2.1]-3-octen-2-one, and then 4-alkoxybicyclo[3.2.1]-3-octen-2-one is hydrolyzed to obtain bicyclo[3.2.1]-3-octane-2,4-dione. In the present application, the intermediate compound III—4-alkoxybicyclo[3.2.1]-3-octen-2-one is generated through the reaction of 3-chlorobicyclo[3.2.1]-3-octen-2-one with a strong alkali and an alcohol, and then a target product bicyclo[3.2.1]-3-octane-2,4-dione can be obtained in high yield through hydrolysis reaction.

### CROSS-REFERENCE TO RELATED APPLICATION

This application is a continuation of international application of PCT application serial no. PCT/CN2023/126283, filed on Oct. 24, 2023, which claims the priority benefit of China application no. 202310042247.9, filed on Jan. 28, 2023. The entirety of each of the above-mentioned patent applications is hereby incorporated by reference herein and made a part of this specification.

### BACKGROUND

### Technical Field

The present application relates to the technical field of medicaments, particularly to a bicyclic ketone compound and a preparation method therefor, and a preparation method for bicyclo[3.2.1]-3-octane-2,4-dione.

### Description of Related Art

Compounds such as bicyclopyrone: 4-hydroxyl-3-{2-[(2-methoxyethoxy)methyl]-6-(trifluoromethyl)-3-pyridylcarbonyl}bicyclo[3.2.1]oct-3-en-2-one, and bicyclic sulcotrione: 3-(2-chloro-4-methylsulfonylbenzoyl)-2-phenylthiobicyclo[3.2.1]oct-2-en-4-one synthesized by bicyclo[3.2.1]-3-octane-2,4-dione (Formula I or Ib) as a common pesticide herbicide synthesis intermediate have certain herbicidal activity.

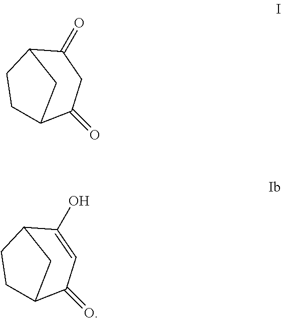 [SMILES: O=C1CC(=O)C2CCC1C2 ; O=C1C=C(O)C2CCC1C2]

Bicyclopyrone has good activity against broadleaf weeds as well as perennial and annual weeds. It can be used in corn, wheat, barley, sugarcane and other crop fields pre- or post-seedling. It provides excellent control of resistant weeds and other recalcitrant broadleaf weeds, especially those that have been resistant to ALS-inhibiting herbicides, growth-hormone herbicides and glyphosate.

Benzobicylon, developed by SDS Biotech K.K., can be absorbed by the roots and stems of weeds and then transported throughout the plant, and it primarily inhibits the biochemical synthesis of carotenoids in photosynthetic pigments, resulting in a decreased content, leaf whitening and eventual weed death. Benzobicylon is highly selective between rice and weeds, safe for rice, highly compatible with the environment, and has low toxicity to fish and mammals. Furthermore, it exhibits a broad herbicidal activity and a long persistence period.

At present, methods for synthesizing such compound shown in formula I (or formula Ib) are as follows:

1) Route A: bicyclo[3.2.1]-6-octen-2,4-dione is obtained through multi-step reactions such as elimination and addition by using 1,1,2,2,3-pentachlorocyclopropane and cyclopentadiene as raw materials, followed by a hydrogenation reaction to generate a compound bicyclo[3.2.1]-3-octane-2,4-dione (W 02005123667).

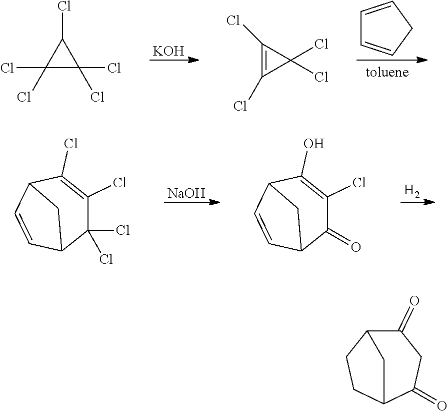

This route has a total yield of 46%, the raw material pentachlorocyclopropane in this route is expensive in price, low in boiling point, large in toxicity and low in product yield, and is not suitable for industrial production.

2) Route B: bicyclo[3.2.1]-3-octane-2,4-dione is synthesized by using 2-norbornanone as a starting raw material (JP10265441(A), JP10265415).

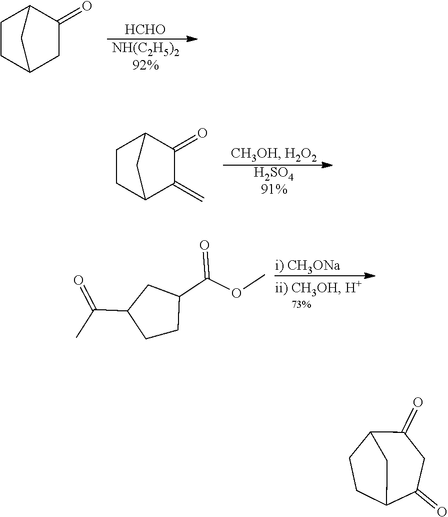

In this route, the product bicyclo[3.2.1]-3-octane-2,4-dione is obtained by using 2-norbornanone as a raw material through Mannich reaction, Bayer Villiger reaction and Claisen condensation reaction. This route has the defects of expensive raw material price, difficult synthesis, low rearrangement reaction conversion rate and the like, and difficultly achieves industrialization.

3) Route C: bicyclo[3.2.1]-3-octane-2,4-dione is obtained by using a cyclopentadiene dimer as a raw material through a 8-step reaction. In this patent, the cyclopentadiene dimer is used as the raw material to generate norbornene or 2-norbornanone, and then the obtained product is subjected to addition, oxidation, ring expansion and rearrangement to obtain bicyclo[3.2.1]-3-octane-2,4-dione (CN1450044A).

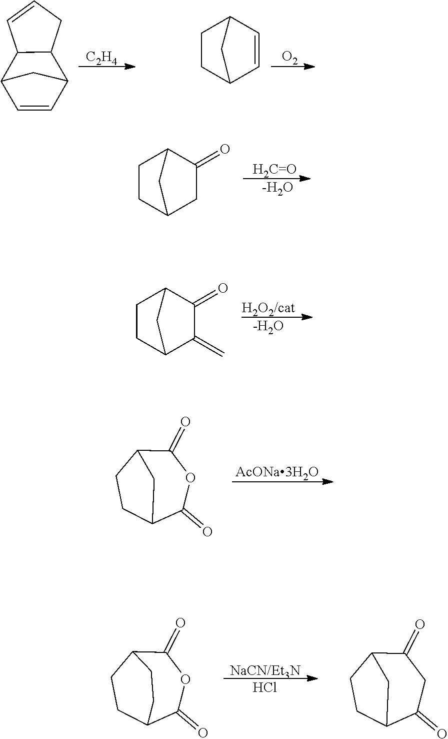

This route uses a petrochemical material as a starting raw material, so it is economic. However, synthesis steps are too complicated and need multi-step reaction. Furthermore, in other patents or documents, bicyclo[3.2.1]-3-octane-2,4-dione has been obtained by synthesis starting from an intermediate norbornene or 2-norbornanone, and therefore the synthesis route has no advantages. The latter half of the reaction process still needs to involve the use of highly toxic cyanide, and there are no many advantages in terms of safety.

4) Route D: bicyclo[3.2.1]-3-octane-2,4-dione is synthesized by using norbornene as a raw material through steps such as carbene ring expansion, hydrolysis, oxidation, cyanation and rehydrolysis (for example CN1440376A and CN105693569A).

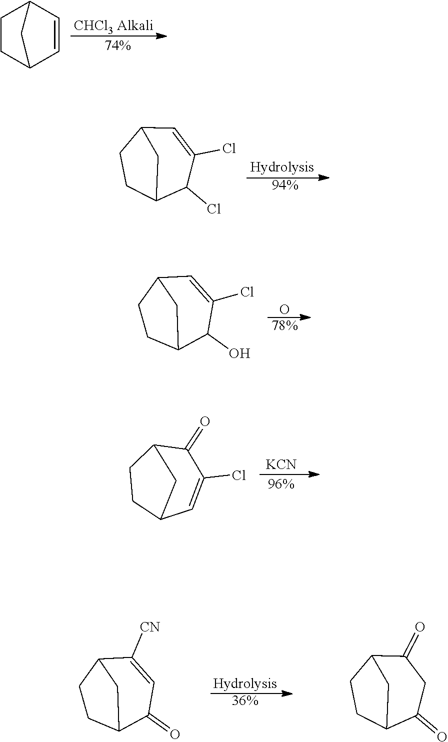

The step-by-step method of the route in CN1440376A has a total yield of 18.75%, uses norbornene as a raw material and undergoes carbene reaction, hydrolysis, oxidation, cyano addition, hydrolysis and other processes. The method has the disadvantages of long route, cumbersome operation and high cyanide dosage, the generation of hydrogen cyanide caused by acidification of the product after hydrolysis and high safety risk, and therefore is not suitable for large-scale industrial production. 3-chlorobicyclo[3.2.1]-3-octen-2-one in this route reacts through a one-pot method to generate bicyclo[3.2.1]-3-octane-2,4-dione, with a maximum yield of 89.2%.

The route in CN105693569A is that potassium cyanide and an aqueous solution of sodium hydroxide are added in a methanol solvent so that 3-chlorobicyclo[3.2.1]-3-octen-2-one reacts through a one-pot method to generate bicyclo[3.2.1]-3-octane-2,4-dione, but the yield is only 84.4%, indicating a low yield.

However, an 4-cyanobicyclo[3.2.1]-3-octen-2-one intermediate is generated through the above two reaction routes. In the above reaction routes, the raw material is 3-chlorobicyclo[3.2.1]-3-octen-2-one, the intermediate is 4-cyanobicyclo[3.2.1]-3-octen-2-one and the product is bicyclo[3.2.1]-3-octane-2,4-dione, alkali hydrolysis can be performed in the presence of sodium hydroxide, so as to result in multiple reaction sites, complex reactions, a large number of byproducts, and further purification needed.

### SUMMARY

### Objective of the Invention

In order to overcome the above defects, the objective of the present application is to provide a bicyclic ketone compound and a preparation method therefor, and a preparation method for bicyclo[3.2.1]-3-octane-2,4-dione.

### Solution

In order to achieve the objective of the present application, the technical solution adopted by the present application is as follows:

In a first aspect, the present application provides a bicyclic ketone compound, which is 4-alkoxybicyclo[3.2.1]-3-octen-2-one with a structural formula represented by Formula III:

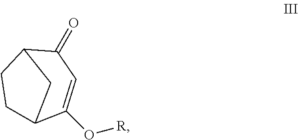

wherein, R is C1-C10 alkyl.

Further, R is C1-C5 alkyl.

Further, R is methyl, ethyl, isopropyl, propyl or butyl.

In a second aspect, provided is a preparation method for the bicyclic ketone compound, comprising the following steps: reacting 3-chlorobicyclo[3.2.1]-3-octen-2-one with an alcohol in the presence of a catalyst and an alkaline substance under an anhydrous condition to generate a compound shown in Formula III.

In a third aspect, provided is a preparation method for bicyclo[3.2.1]-3-octane-2,4-dione, comprising the following steps:
reacting 3-chlorobicyclo[3.2.1]-3-octen-2-one with an alcohol in the presence of a catalyst and a strong alkali under an anhydrous condition to generate 4-alkoxybicyclo[3.2.1]-3-octen-2-one, and then conducting hydrolysis to obtain bicyclo[3.2.1]-3-octane-2,4-dione.

Further, the catalyst is selected from cyanides, or the catalyst is one or more selected from the group consisting of potassium cyanide, sodium cyanide, zinc cyanide, nickel cyanide, copper cyanide and acetone cyanohydrin;
and/or, a molar ratio of 3-chlorobicyclo[3.2.1]-3-octen-2-one to the catalyst is 100:1-40, or 100:1-30, or 100:1-20, or 100:1-10, or 100:3-7, or 100:5.

Further, a molar ratio of 3-chlorobicyclo[3.2.1]-3-octen-2-one to the strong alkali is 1:1-10, or 1:1-8, or 1:1.5-5, or 1:2-5, or 1:3.

Further, the alkaline substance or the strong alkali is one or more selected from the group consisting of alkali metal hydroxide, alkali earth metal hydroxide and alcohol alkali; alternatively, the strong alkali is one or more selected from the group consisting of sodium hydroxide, potassium hydroxide, lithium hydroxide, calcium hydroxide, magnesium hydroxide, sodium methoxide, sodium ethoxide, potassium methoxide and potassium ethoxide.

When the intermediate compound shown in formula III is generated, a strong alkali needs to be used. Weak alkali is difficult for the raw material to generate the intermediate compound, or causes a low yield. Further, the alcohol is one or more of methanol, ethanol, propanol, isopropanol, propylene glycol, butanol and pentanol.

And/or, a mass ratio of 3-chlorobicyclo[3.2.1]-3-octen-2-one to the alcohol is 1:1-20, or 1:1-10, or 1:1-5.

The mass ratio of 3-chlorobicyclo[3.2.1]-3-octen-2-one to the alcohol is adjusted along with changes in reaction temperature, for example:
when the reaction temperature is a refluxing temperature of a solvent, the mass ratio of 3-chlorobicyclo[3.2.1]-3-octen-2-one to the alcohol can be 1:20.

When the reaction temperature is low, for example the reaction temperature is 40-60° C., or between 40° C. and the refluxing temperature of the solvent, the use amount of methanol can be reduced, which is conducive to the proceeding of the reaction. Preferably, the mass ratio of 3-chlorobicyclo[3.2.1]-3-octen-2-one to the alcohol is 1:1-5.

Further, the reaction is carried out in a solvent; the solvent is a polar non-protonic solvent or a polar protonic solvent; or, the solvent is one or more selected from the group consisting of methanol, ethanol, propanol, isopropanol, toluene, xylene, methyl tert-butyl ketone, butyl acetate, dichloromethane and dichloroethane. In the reaction, the alcohol can be used as both a reaction raw material and a reaction solvent. In some embodiments, when toluene is used as the solvent, the yield may be reduced, and the solvent is one or more selected from the group consisting of methanol, ethanol, propanol, isopropanol, methyl tert-butyl ketone, butyl acetate, dichloromethane and dichloroethane.

Further, the hydrolysis is acid hydrolysis or acid adjustment after alkali hydrolysis.

Further, the acid used for acid hydrolysis is one or more of hydrochloric acid, sulfuric acid, phosphoric acid, p-methylbenzenesulfonic acid, acetic acid and trifluoroacetic acid.

Further, a molar ratio of alkoxybicyclo[3.2.1]-3-octen-2-one to the acid is 1:1-10, or 1:1-8, or 1:1-5.

Further, the alkali used for alkali hydrolysis is alkali metal hydroxide, alcohol alkali or alkali metal carbonate; alternatively, the alkali used for alkali hydrolysis is one or more of sodium hydroxide, potassium hydroxide, lithium hydroxide, potassium carbonate, sodium carbonate, sodium methoxide and sodium ethoxide.

Further, a molar ratio of 3-chlorobicyclo[3.2.1]-3-octen-2-one to the alkali is 1:1-10, or 1:1-8, or 1:1.5-5, or 1:2-5, or 1:3.

Further, in the reaction of 3-chlorobicyclo[3.2.1]-3-octen-2-one with an alcohol, the reaction temperature is 0° C. to a refluxing temperature of each solvent, or 30° C. to a refluxing temperature of each solvent, or 40° C. to a refluxing temperature of each solvent, or 50° C. to a refluxing temperature of each solvent, or 60° C. to a refluxing temperature of each solvent, or a refluxing temperature of each solvent.

In some embodiments, when the reaction temperature is reduced, it is needed to reduce the use amount of the alcohol and/or solvent so that the raw materials more easily reacted.

Further, the materials after 3-chlorobicyclo[3.2.1]-3-octen-2-one reacts with an alcohol are treated by using any one of the following methods in 1) and 2):
1) extraction and liquid separation is performed on the materials after 3-chlorobicyclo[3.2.1]-3-octen-2-one reacts with an alcohol for at least once to obtain a crude oil product, and then conducting hydrolysis; wherein preferably, extraction and liquid separation is carried out twice, which can more effectively remove cyano radicals; and
2) after 3-chlorobicyclo[3.2.1]-3-octen-2-one reacts with an alcohol, a solvent is removed by evaporation, water is supplemented to continue the reaction (alkali hydrolysis). Since the reaction system contains alkali, alkali hydrolysis can occur after the addition of water; further, in the method 2), after water is supplemented to continue the reaction, acid adjustment is performed to obtain bicyclo[3.2.1]-3-octane-2,4-dione.

The reaction route of the present application is divided into a two-step method and a one-pot method, which specifically are as follows:

I) In the two-step method of the present application, 3-chlorobicyclo[3.2.1]-3-octen-2-one reacts with an alkali and an alcohol under an anhydrous condition in the presence of a cyanide catalyst to generate an intermediate compound III. Cyano radicals are removed through extraction and liquid separation, an oily product is subjected to alkali hydrolysis and then undergoes acid adjustment or acid hydrolysis to obtain a target product:

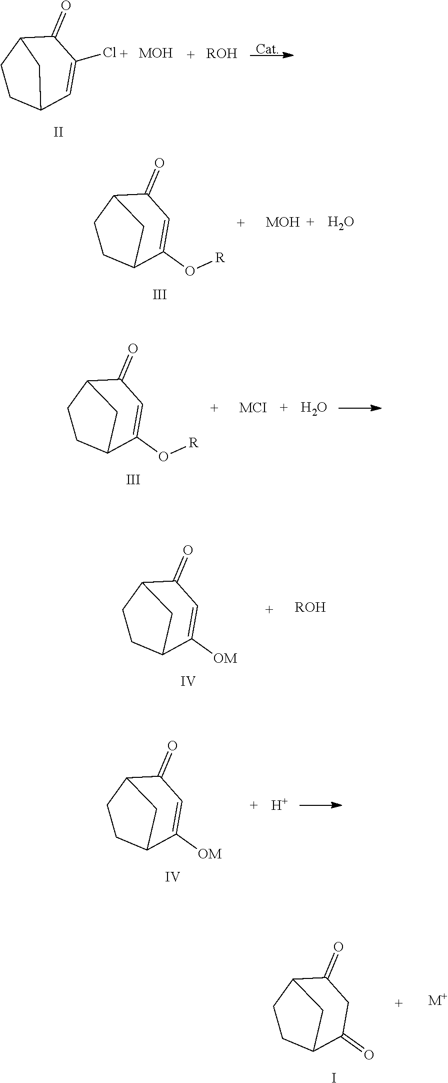

(R=methyl, ethyl, isopropyl, propyl, butyl, etc. M=N a, K, Li, Mg, etc.)

When the intermediate compound III is generated, it is needed to use a strong alkali and an alcohol, and weak alkali is difficult for the raw materials to generate the intermediate compound III, or causes a low yield.

A molar ratio of 3-chlorobicyclo[3.2.1]-3-octen-2-one to the catalyst is 1:1-10, or 1:1-8, or 1:1.5-5, or 1:2-5, or 1:3.

In the reaction of 3-chlorobicyclo[3.2.1]-3-octen-2-one with an alcohol, the reaction temperature is 0° C. to a refluxing temperature of each solvent, or 30° C. to a refluxing temperature of each solvent, or 40° C. to a refluxing temperature of each solvent, or 50° C. to a refluxing temperature of each solvent, or 60° C. to a refluxing temperature of each solvent, or a refluxing temperature of each solvent.

When the intermediate of formula III (4-alkoxybicyclo[3.2.1]-3-octen-2-one) is generated, a catalytic amount (a small amount) of a cyano agent is used, after extraction and liquid separation, the content of cyano radicals in an organic product is in PPM (parts per million) level, and most of the cyano radicals exist in an aqueous phase, so the cyano reaction relative to that in CN1440376A has higher safety and economy. When the intermediate of formula III of the present application is hydrolyzed, a target product of Formula I can be obtained by using acid adjustment after alkali hydrolysis or acid hydrolysis. Acid hydrolysis has the advantages that the final target product bicyclo[3.2.1]-3-octane-2,4-ditone can be directly obtained, but after the reaction is ended, an organic solvent needs to be used for extraction and liquid separation, and concentration and spin drying are needed to obtain the solid target product. The acid hydrolysis method has fewer reaction steps, simple operation, high reaction yield, and high product content. The alkali hydrolysis has the advantages that hydrochloric acid is added to adjust the pH after alkali hydrolysis. During the adjustment process, the product bicyclo[3.2.1]-3-octane-2,4-dione can directly precipitate out from an aqueous phase, and solid products can be directly obtained after suction filtration, and therefore alkali hydrolysis is simple in operation, high in content and high in yield.

Relative to the two-step method of the reaction route in CN1440376A, the two-step method of the present application has the advantages: during the hydrolysis, side reaction competition is few with no obvious byproducts, synthesis operation is relatively simple, acid hydrolysis and alkali hydrolysis individually have their own advantages, the yield can be significantly improved, and the production of hydrogen cyanide can be effectively avoided. The problems in the existing technical routes in CN1440376A and the like that most of the side reactions are in competition, many byproducts are generated, and toxic gases such as hydrogen cyanide are easily generated to cause hazards are solved. In the first step of the step-by-step method of CN1440376A, a product after extraction, pickling and drying after cyano reactions contains many byproducts (a gas phase spectrogram is shown in FIG. 12), and in the second step of the step-by-step method of CN1440376A, a product after acidification, extraction and drying after hydrolysis reaction contains many byproducts (a gas phase spectrogram is shown in FIG. 14). The two-step method in the present application has higher product purity and fewer byproducts (the first step generates the methoxy intermediate, and the gas chromatography of the product after the hydrolysis in the second step can be seen in FIG. 15 and FIG. 16).

II) In the one-pot method route of the present application, 3-chlorobicyclo[3.2.1]-3-octen-2-one is used as a main reaction raw material for synthesis; in the presence of a catalyst and an alkaline substance (usually a strong alkali), it reacts with an alcohol in a solvent to obtain the compound of formula III. After the reaction is ended, the solvent is removed by distillation, and water is added to continue the reaction (alkali hydrolysis). After the reaction is completed, bicyclo[3.2.1]-3-octane-2,4-dione (Formula I or Formula Ib) is obtained by acidification.

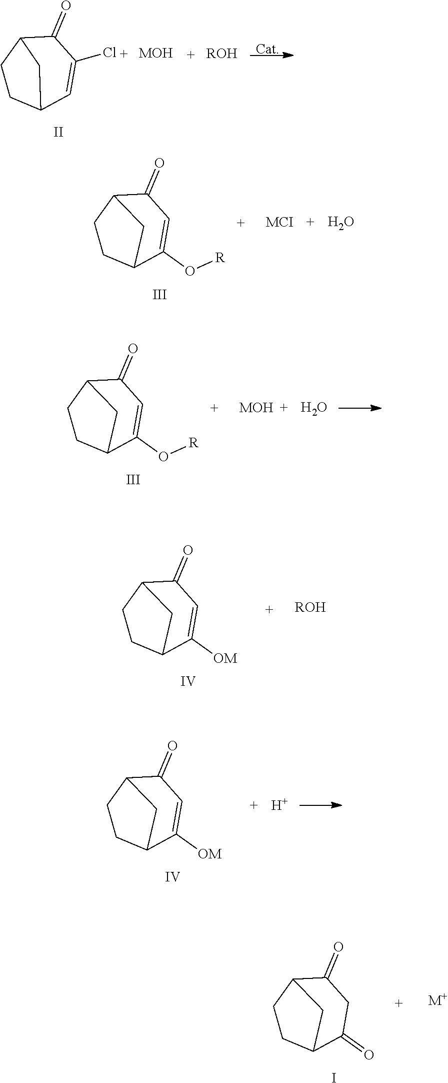

When the intermediate compound of formula III is generated, it is needed to use a strong alkali and an alcohol, weak alkali is difficult for the raw material to generate the intermediate compound of formula III, or causes a low yield.

A molar ratio of 3-chlorobicyclo[3.2.1]-3-octen-2-one to the alkali is 1:1-10, or 1:1-8, or 1:1.5-5, or 1:2-5, or 1:3.

In the reaction of 3-chlorobicyclo[3.2.1]-3-octen-2-one with an alcohol, the reaction temperature is 0° C. to a refluxing temperature of each solvent, or 30° C. to a refluxing temperature of each solvent, or 40° C. to a refluxing temperature of each solvent, or 50° C. to a refluxing temperature of each solvent, or 60° C. to a refluxing temperature of each solvent, or a refluxing temperature of each solvent.

The production route of the one-pot method of the present application does not undergo intermediate separation and purification (extraction and liquid separation), with high reaction yield and few operational steps, and has good applicability in industrial production. In the one-pot method of the present application, a compound of Formula III (4-alkoxybicyclo[3.2.1]-3-octen-2-one) is firstly synthesized. This reaction is not prone to producing byproducts (a gas phase spectrum of a product obtained after precipitation, washing and drying is shown in FIG. 18, with almost no impurity peaks except for a target product), the product obtained after precipitation, washing and drying is subjected to subsequent reactions, which can effectively improve production efficiency. Compared to the existing technology patents such as CN1440376A, most of the byproducts need the production route of the one-pot method for further purification, (a gas phase spectrum of a product obtained after extraction, drying, and concentration is shown in FIG. 17, which contains impurity peaks in addition to a target product), the one-pot method of the present application has significantly improved production efficiency and economy.

Compared with the two-step method, the one-pot method in the present application can achieve continuous reaction and relatively simple processing, reduced separation losses and improved reaction yield. However, there are small amounts of cyanide compounds in the reaction system, resulting in low product content and the presence of some byproducts. The advantage of the two-step method is that the byproducts generated in each step of the reaction can be removed, resulting in a decrease in yield, but the product content is high, which is beneficial for subsequent reactions.

### Beneficial Effects

In the present application, 3-chlorobicyclo[3.2.1]-3-octen-2-one reacts with an alcohol in the presence of a catalyst by using a strong alkali to generate an intermediate compound of formula III—4-alkoxybicyclo[3.2.1]-3-octen-2-one. Through hydrolysis reaction, the target product bicyclo[3.2.1]-3-octen-2,4-dione can be obtained in high yield. The specific preparation methods can be divided into a two-step method or a one-pot method to prepare bicyclo[3.2.1]-3-octane-2,4-dione. In the two-step method, 4-alkoxybicyclo[3.2.1]-3-octen-2-one is generated. After the solvent is removed through distillation, the organic solvent is subjected to extraction and liquid separation to completely separate the generated product from a catalytic amount of cyano radicals. The cyano radicals in wastewater are separately quenched to ensure the safety of the production process. The synthesis method of the present application has simple operation, low dosage of highly toxic chemicals and greatly improved reaction safety, and is conducive to industrial production safety and environmental protection wastewater treatment. In the one-pot method of the present application, the intermediate generated is 4-alkoxybicyclo[3.2.1]-3-octen-2-one. When the intermediate is hydrolyzed in the one-pot method, there is no side reaction competition, the reaction yield is improved, and the production efficiency and economy are greatly improved.

### BRIEF DESCRIPTION OF THE DRAWINGS

One or more examples are exemplified by the figures in the accompanying drawings that correspond thereto and the exemplifications are not intended to limit to the examples. As used herein, the word “exemplification” means “serving as an example, embodiment, or illustrative”. Any example described herein as “exemplification” is not necessarily to be construed as superior to or better than other examples.

FIG. 1 is a nuclear magnetic resonance hydrogen spectrum of an analytical sample (4-cyanobicyclo[3.2.1]-3-octen-2-one) of the present application.

FIG. 2 is a nuclear magnetic resonance carbon spectrum of an analytical sample (4-cyanobicyclo[3.2.1]-3-octen-2-one) of the present application.

FIG. 3 is a nuclear magnetic resonance hydrogen spectrum of an analytical sample (4-methoxybicyclo[3.2.1]-3-octen-2-one) of example 1 of the present application.

FIG. 4 is a nuclear magnetic resonance carbon spectrum of an analytical sample (4-methoxybicyclo[3.2.1]-3-octen-2-one) of example 1 of the present application.

FIG. 5 is a nuclear magnetic resonance hydrogen spectrum of an analytical sample (4-ethoxybicyclo[3.2.1]-3-octen-2-one) of example 2 of the present application.

FIG. 6 is a nuclear magnetic resonance carbon spectrum of an analytical sample (4-ethoxybicyclo[3.2.1]-3-octen-2-one) of example 2 of the present application.

FIG. 7 is a nuclear magnetic resonance hydrogen spectrum of an analytical sample (4-isopropoxybicyclo[3.2.1]-3-octen-2-one) of example 3 of the present application.

FIG. 8 is a nuclear magnetic resonance carbon spectrum of an analytical sample (4-isopropoxybicyclo[3.2.1]-3-octen-2-one) of example 3 of the present application.

FIG. 9 is a nuclear magnetic resonance hydrogen spectrum of an analytical sample (bicyclo[3.2.1]-3-octane-2,4-dione) of example 20 of the present application.

FIG. 10 is a nuclear magnetic resonance carbon spectrum of an analytical sample (bicyclo[3.2.1]-3-octane-2,4-dione) of example 20 of the present application.

FIG. 11 shows a gas phase spectrum of a product at the beginning of the reaction in step 1) of comparative example 1 after undergoing extraction, acid washing and drying. Wherein, 3-chlorobicyclo[3.2.1]-3-octen-2-one is generated at 13.46 min, and impurities are generated at 14.86 min.

FIG. 12 shows a gas phase spectrum of a product after the reaction in step 1) of comparative example 1 after undergoing extraction, acid washing and drying. Wherein, 4-cyanobicyclo[3.2.1]-3-octen-2-one intermediate is generated at 13.55 min, and impurities are at other peaks.

FIG. 13 is a gas phase spectrum of a product when the hydrolysis reaction of step 2) of comparative example 1 after undergoing acid adjustment, extraction and drying for 1 hour. Wherein, 4-cyanobicyclo[3.2.1]-3-octen-2-one intermediate is generated at 13.51 min, and impurities are at 14.86 min.

FIG. 14 is a gas phase spectrum of a product after the hydrolysis reaction of step 2) of comparative example 1 after undergoing acid adjustment, extraction and drying. Wherein, 4-cyanobicyclo[3.2.1]-3-octen-2-one intermediate is generated at 13.51 min, which almost disappears, and impurities are at other peaks.

FIG. 15 is a gas phase spectrum of an oily crude product in example 1 of the present application. Wherein, 4-methoxybicyclo[3.2.1]-3-octen-2-one intermediate is generated at 12.90 min.

FIG. 16 is a gas phase spectrum of a product of example 20 of the present application. Wherein, bicyclo[3.2.1]-3-octen-2,4-dione intermediate is generated at 12.46 min.

FIG. 17 is a gas phase spectrum of a product of comparative example 2. Wherein, a bicyclo[3.2.1]-3-octen-2,4-dione product is generated at 12.43 min, and impurities are at other peaks.

FIG. 18 is a gas phase spectrum of a light yellow solid product of example 33A of the present application. Wherein, a target product bicyclo[3.2.1]-3-octane-2,4-dione is generated at 12.43 min.

### DESCRIPTION OF THE EMBODIMENTS

In order to make the purpose, technical solutions and advantages of the present application clearer, the technical solutions in the examples of the present application will be described clearly and completely. Obviously, the described examples are some of the examples of the present application, but not all of them. Based on the examples of the present invention, all other examples obtained by those of ordinary skill in the art without creative work are falling within the protection scope of the present invention.

In addition, in order to better explain the present application, a lot of specific details are given in the following examples. It will be understood by those skilled in the art that the present application may be implemented without certain specific details. In some examples, materials, solutions, methods, means, etc., well known to those skilled in the art, are not described in detail so as to highlight the spirit of the present application.

Throughout the specification and claims, the term “comprising” or variations thereof, such as “including” or “containing”, will be understood to include the stated components and not to exclude other elements or other components, unless expressly indicated otherwise.

The contents of products in the following examples are confirmed by a liquid or gas chromatograph, the tracking in the reaction process employs an area normalization method, and the purity of the reaction product employs an external standard method.
LCMS: Liquid chromatography mass spectrometry.
GCMS: Gas chromatography mass spectrometry.
HPLC: High Performance Liquid Chromatography.
GC: Gas chromatography.
NMR: Nuclear magnetic resonance spectrometry.

3-chlorobicyclo[3.2.1]-3-octen-2-one in the following examples can be self-made or commercially available; if not specified, the reaction process and results are detected by high-pressure liquid chromatography or gas phase chromatography.

In the existing technology CN1440376A, a two-step method and a one-pot method are used respectively:

1) In the two-step method of CN1440376A, cyano reaction A) and hydrolysis reaction B) are carried out respectively:

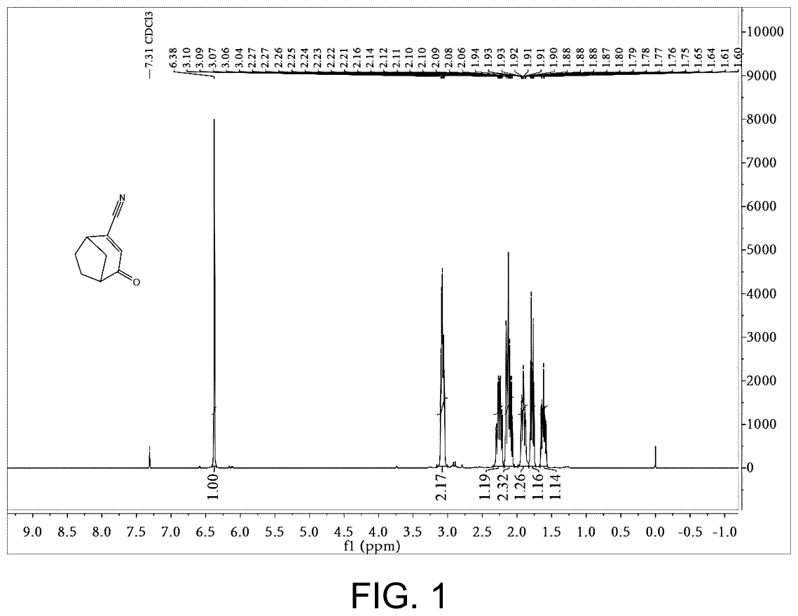

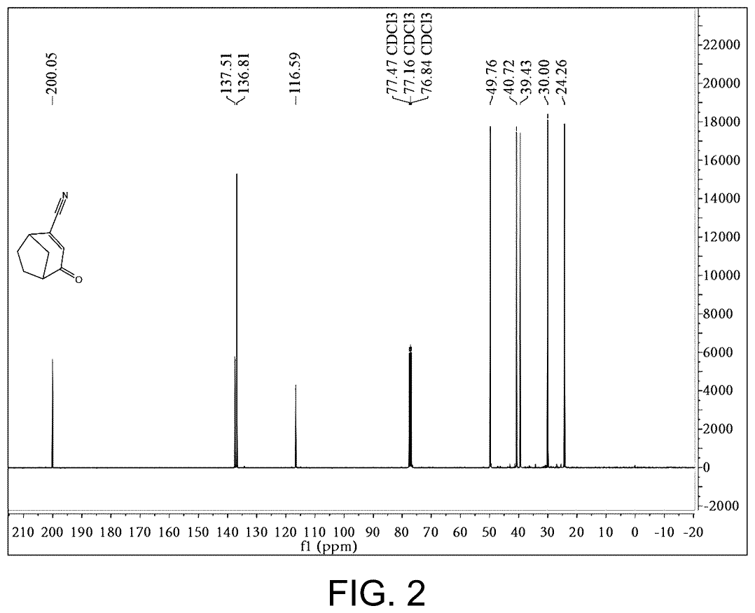

A) For the cyano reaction of CN1440376A, in terms of the selection of the synthesis route for compound II, equivalent levels of acetone cyanohydrin are selected to be added for cyano reaction to obtain Formula V 4-cyanobicyclo[3.2.1]-3-octen-2-one. The nuclear magnetic resonance image is shown in FIGS. 1 and 2:

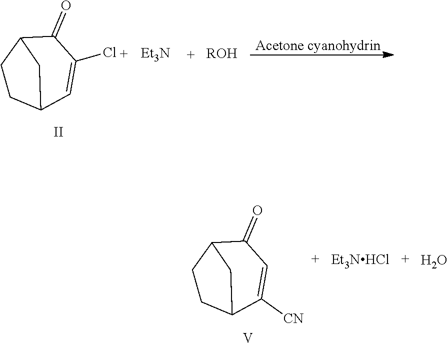

¹H—NMR (400 MHz, CDCl₃, δ/ppm) 6.38 (s, 1H), 3.07 (dt, J=11.9, 5.5 Hz, 2H), 2.34˜2.19 (m, 1H), 2.18˜2.03 (m, 2H), 1.99˜1.83 (m, 1H), 1.78 (dt, J=12.0, 4.5 Hz, 1H), 1.67˜1.55 (m, 1H). ¹³C-NMR (100 MHz, CDCl₃, δ/ppm) 200.05, 137.51, 136.81, 116.59, 77.47, 77.16, 76.84, 49.76, 40.72, 39.43, 30.00, 24.26.

In this cyano reaction of CN1440376A, triethylamine was used as an alkali and methanol as a solvent. After the reaction is ended, the reaction solution is poured into water and then extracted with ethyl acetate. And an organic phase is washed with hydrochloric acid. 4-cyanobicyclo[3.2.1]-3-octen-2-one is obtained, with a yield of 85% (see example A, step d, CN1440376A). Although extraction can be carried out after the cyano reaction, since the molar amount of acetone cyanohydrin is equivalent to that of the raw material 3-chlorobicyclo[3.2.1]-3-octen-2-one with a large usage amount, a small amount of unreacted acetone cyanohydrin has a certain residue in the organic phase, and therefore when the organic phase needs to be washed with 2N hydrochloric acid, a small amount of acetone cyanohydrin or cyanide byproducts may react with hydrochloric acid to generate highly toxic gas hydrogen cyanide, which poses a certain safety risk.

Furthermore, this reaction can only be performed at a lower reaction temperature (25° C.). When the reaction temperature is raised, more byproducts are produced—the color of the reaction system is darker than that at room temperature, and tar-like substances appear, making it more viscous overall. When the reaction temperature is below 25° C., the reaction rate greatly decreases, and the reaction time needs to be extended. Moreover, there is ultimately some surplus of raw materials, resulting in incomplete reaction.

B) The hydrolysis reaction of CN1440376A is as follows: Formula V 4-cyanobicyclo[3.2.1]-3-octen-2-one is subjected to alkali hydrolysis with a sodium hydroxide solution, the generated 4-hydroxysodium bicyclo[3.2.1]-3-octen-2-one is acidified by dropwise addition of hydrochloric acid to form a target product bicyclo[3.2.1]-3-octane-2,4-dione, and then the target product bicyclo[3.2.1]-3-octane-2,4-dione is extracted with ethyl acetate and concentrated to obtain a product. In the patent example, the yield of this step is only 36% (see example A, step e, CN1440376A).

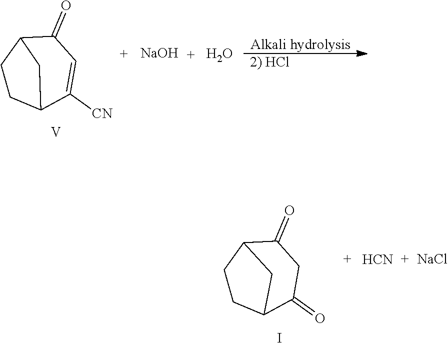

After the hydrolysis reaction of CN1440376A, when in acid adjustment, the generated hydrogen cyanide is still partially present in the reaction system after being absorbed by an absorption bottle, which may lead to the formation of amide or carboxylic acid byproducts from cyano radicals and the product under an alkaline condition and high temperatures, resulting in a decrease in reaction yield. Under the condition that water is chosen as a solvent for this reaction, a phase transfer catalyst (such as tetrabutylammonium chloride) needs to be added to improve the solubility of the cyanide catalyst, which increases the raw material cost of the route.

The hydrolysis step of CN1440376A has a relatively low yield. By research, the inventors of the present application believe that the reason for low yield is that there are many side reactions, which forms competition and generates more byproducts. For example, the cyano undergoes alkali hydrolysis and then acidification under an alkaline condition, which may generate corresponding carboxylic acid products. For example, in literature Yasuyo S, Nadaraj P, Kevin A R, Hiroyuki O, Biosynthesis of phonomics: cyclohexanecarboxylic acid as the starter unit [J]. Tetrahedron, 2003, 59 (38): 7465-7471, under the conditions of potassium hydroxide and ethanol, cyclohexane nitrile undergoes a reflux reaction, followed by acidification extraction, to obtain cyclohexane carboxylic acid with a yield of about 54%. The hydrolysis conditions of CN1440376A are similar to those in this literature, and carboxylic acid hydrolysis byproducts corresponding to 4-cyanobicyclo[3.2.1]-3-octen-2-one may also be generated. For hydrolysis under an alkaline condition, cyano is hydrolyzed to carboxylate, competes with the hydroxyl salts generated by substitution, which is an important reason that the hydrolysis reaction of CN1440376A has a low yield.

In the two-step method of CN1440376A, the first-step reaction has drawbacks of generating highly toxic gas (pickling generates hydrogen cyanide) and cumbersome operation. The hydrolysis reaction in the second step has the problems of many byproducts and low yield. When the 4-cyanobicyclo[3.2.1]-3-octen-2-one intermediate is hydrolyzed, cyano radicals are released, and a large amount of highly toxic hydrogen cyanide gas is generated during the acid adjustment to cause great safety hazards. The entire reaction route has drawbacks of many byproducts, low reaction yield, and a large amount of hydrogen cyanide produced during the acid adjustment.

2) In the one-pot method of CN1440376A, 3-chlorobicyclo[3.2.1]-3-oct-2-one, sodium hydroxide and a potassium cyanide solid are subjected to a refluxing reaction in methanol. After acid adjustment, ethyl acetate is used for extraction, liquid separation and concentration to obtain a target product bicyclo[3.2.1]-3-octane-2,4-dione.

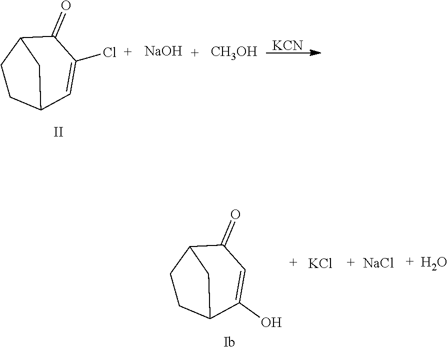

In the one-pot method of CN1440376A, although the yield can reach 89.2%, the intermediate 4-cyanobicyclo[3.2.1]-3-octen-2-one generated in the one-pot method can undergo cyano hydrolysis under an alkaline condition to generate 4-carboxylic bicyclo[3.2.1]-3-octen-2-one. This side reaction competes with the main reaction to result in a decrease in reaction yield. This hydrolysis reaction is caused by its reaction mechanism, and the further improvement of the yield is difficultly achieved through condition optimization.

The reaction route of the present application is also divided into a two-step method and a one-pot method, which specifically are as follows:

I) in the two-step method of the present application, 3-chlorobicyclo[3.2.1]-3-octen-2-one reacts with an alkali and an alcohol under an anhydrous condition in the presence of a cyanide catalyst to generate an intermediate compound III (4-alkoxybicyclo[3.2.1]-3-octen-2-one). Cyano radicals are removed through extraction and liquid separation, an oily product is subjected to alkali hydrolysis and then undergoes acid adjustment or acid hydrolysis to obtain a target product:

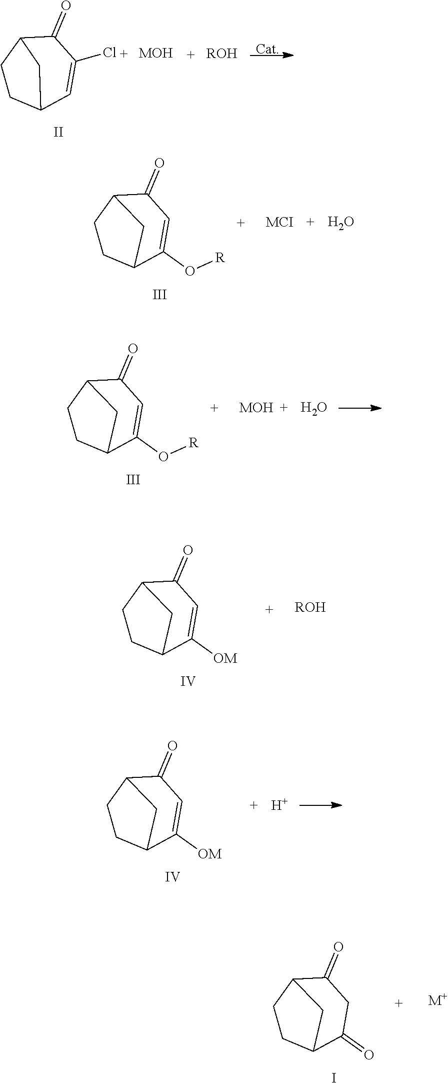

When the intermediate III (4-alkoxybicyclo[3.2.1]-3-octen-2-one) is generated, a catalytic amount (a small amount) of a cyano agent is used, after extraction and liquid separation, the content of cyano radicals in an organic product is in PPM (parts per million) level, and most of the cyano radicals exist in an aqueous phase, so the cyano reaction relative to that in CN1440376A has higher safety and economy. When the intermediate III of the present application is hydrolyzed, a target product of Formula I can be obtained by using acid adjustment or acid hydrolysis after alkali hydrolysis. Acid hydrolysis has the advantages that the final target product bicyclo[3.2.1]-3-octane-2,4-ditone can be directly obtained, but after the reaction is ended, an organic solvent needs to be used for extraction and liquid separation, and concentration and spin drying are needed to obtain the solid target product. The acid hydrolysis method has fewer reaction steps, simple operation, high reaction yield, and high product content. The alkali hydrolysis has the advantages that hydrochloric acid is added to adjust the pH after alkali hydrolysis. During the adjustment process, the product bicyclo[3.2.1]-3-octane-2,4-dione can directly precipitate out from an aqueous phase, and solid products can be directly obtained after suction filtration, and therefore alkali hydrolysis is simple in operation, high in content and high in yield.

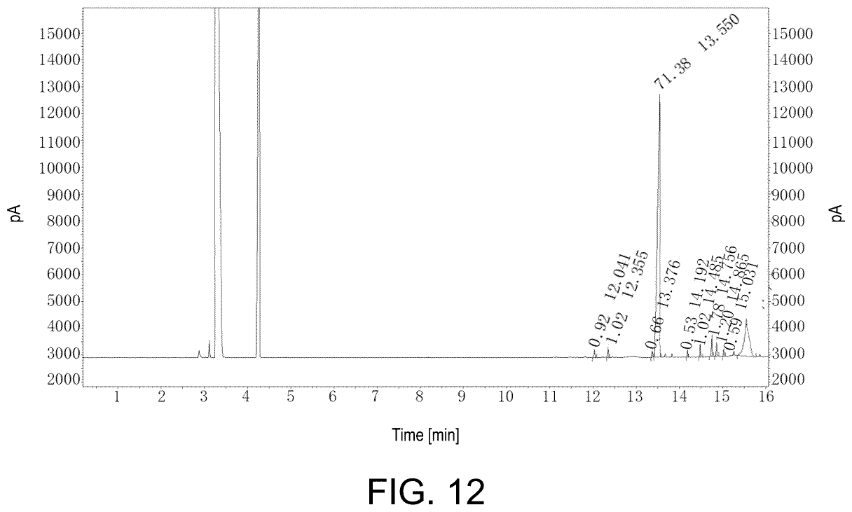

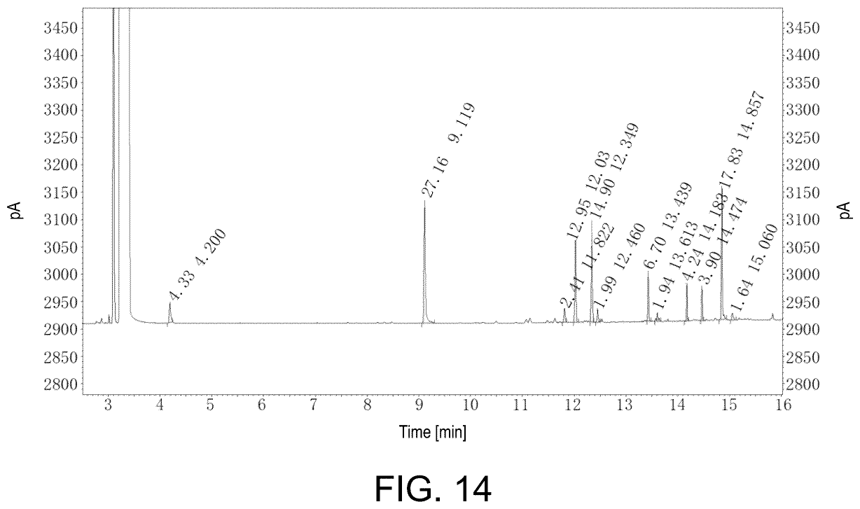

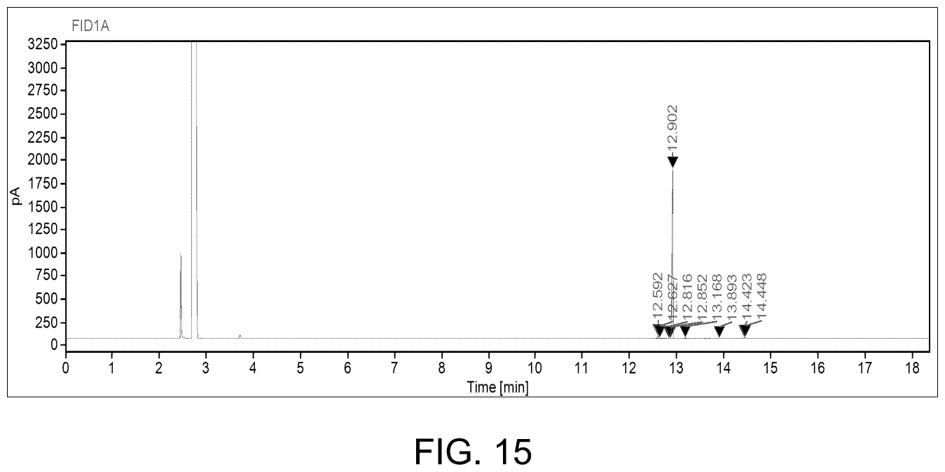

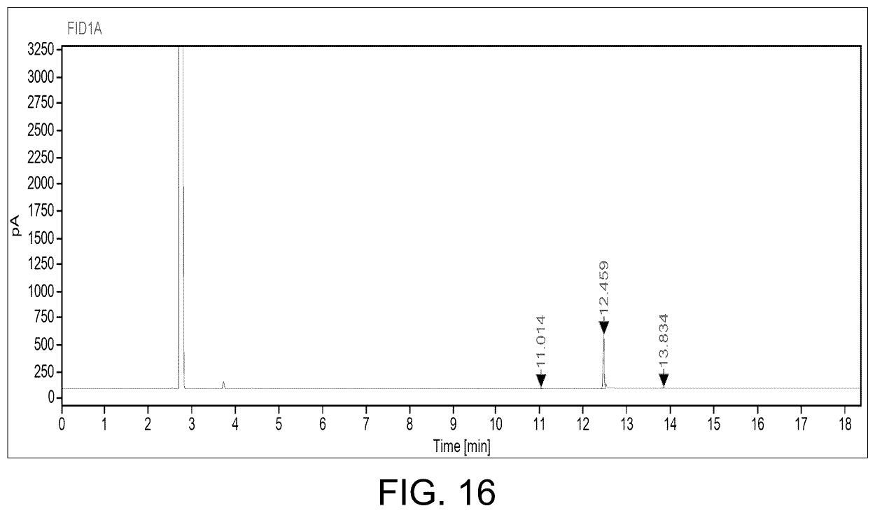

Relative to the two-step method of the reaction route in CN1440376A, the two-step method of the present application has the advantages: during the hydrolysis, side reaction competition is few with no obvious byproducts, synthesis operation is relatively simple, acid hydrolysis and alkali hydrolysis individually have their own advantages, the yield can be significantly improved, and the production of hydrogen cyanide can be effectively avoided. The problems in the existing technical routes in CN1440376A and the like that most of the side reactions are in competition, many byproducts are generated, and toxic gases such as hydrogen cyanide are easily generated so as to cause hazards are solved. In the first step of the step-by-step method of CN1440376A, a product after extraction, pickling and drying post cyano reactions contains many byproducts (a gas phase spectrogram is shown in FIG. 12), and in the second step of the step-by-step method of CN1440376A, a product after acidification, extraction and drying after hydrolysis reaction contains many byproducts (a gas phase spectrogram is as shown in FIG. 14). The two-step method in the present application has higher product purity and fewer byproducts (the first step generates the methoxy intermediate, and the gas chromatography of the product after the hydrolysis in the second step can be seen in FIG. 15 and FIG. 16).

II) In the one-pot method route of the present application, 3-chlorobicyclo[3.2.1]-3-octen-2-one is used as a main reaction raw material for synthesis; in the presence of a catalyst and an alkaline substance, it reacts with an alcohol in a solvent to obtain formula III. After the reaction is ended, the solvent is removed by distillation, and water is added to continue the reaction (alkali hydrolysis). After the reaction is completed, bicyclo[3.2.1]-3-octane-2,4-dione (Formula I or Formula Ib) is obtained by acidification.

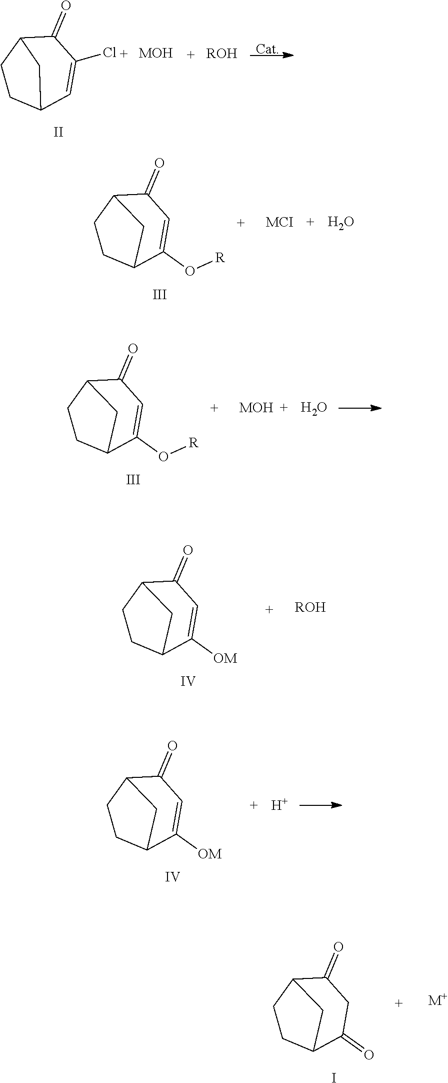

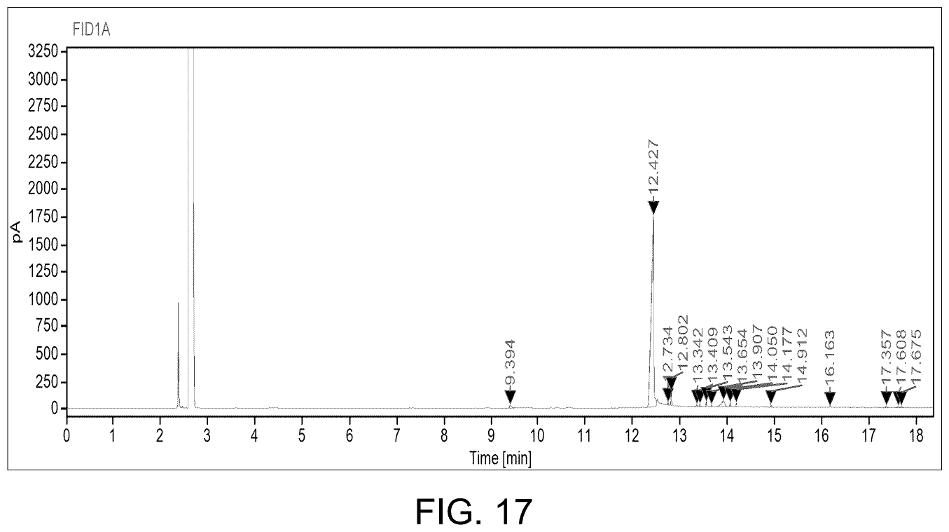

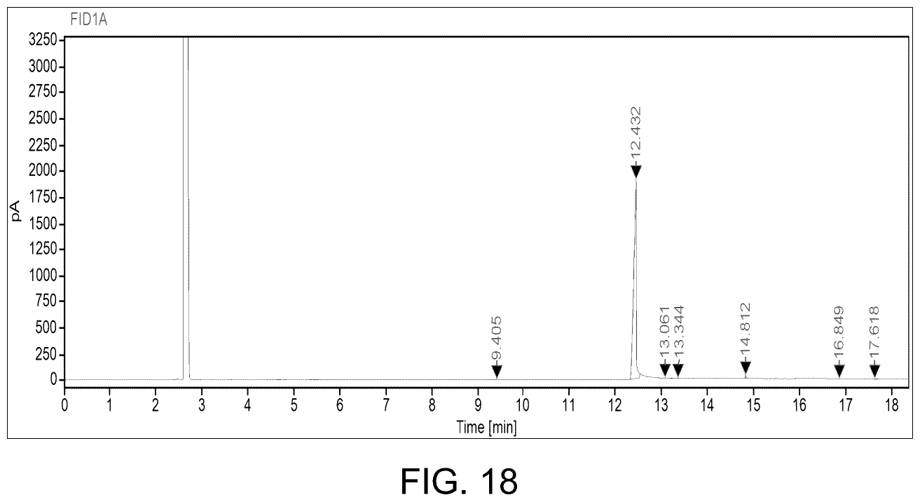

The production route of the one-pot method of the present application does not undergo intermediate separation and purification (extraction and liquid separation), with high reaction yield and few operational steps, and has good applicability in industrial production. In the one-pot method of the present application, a compound of Formula III (4-alkoxybicyclo[3.2.1]-3-octen-2-one) is firstly synthesized, this reaction is not prone to producing byproducts (a gas phase spectrum of a product obtained after precipitation, washing and drying is shown in FIG. 18, with almost no impurity peaks except for a target product), the product obtained after precipitation, washing and drying is subjected to subsequent reactions, which can effectively improve production efficiency. Compared to the existing technology patents such as CN1440376A, most of the byproducts need the production route of the one-pot method for further purification, (a gas phase spectrum of a product obtained after extraction, drying, and concentration is shown in FIG. 17, which contains impurity peaks in addition to a target product), the one-pot method of the present application has significantly improved production efficiency and economy.

Compared with the two-step method, the one-pot method in the present application can achieve continuous reaction and relatively simple processing, reduced separation losses and improved reaction yield. However, there are small amounts of cyanide compounds in the reaction system, resulting in low product content and the presence of some byproducts. The advantage of the two-step method is that the byproducts generated in each step of the reaction can be removed, resulting in a decrease in yield, but the product content is high, which is beneficial for subsequent reactions.

The target product belongs to 1,3-dione compounds, and the methylene has strong acidity under the joint influence of two carbonyl groups, and has tautomerism, such as Formulas I and Ib.

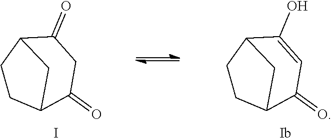

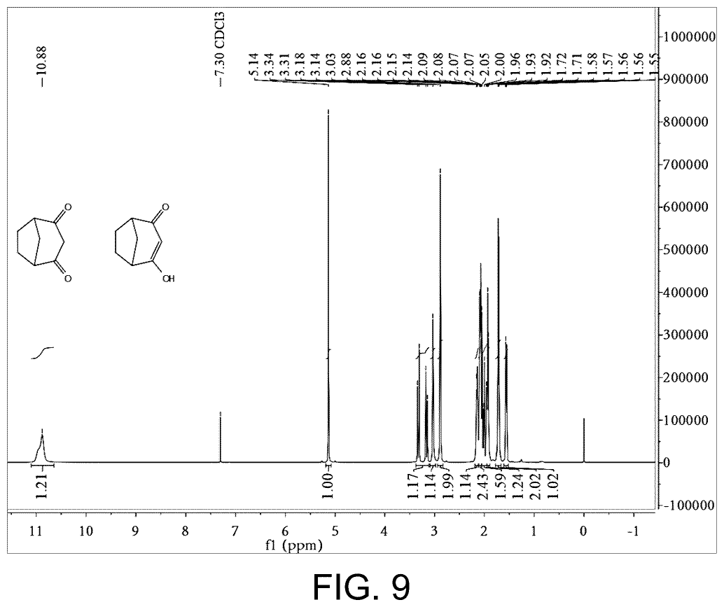

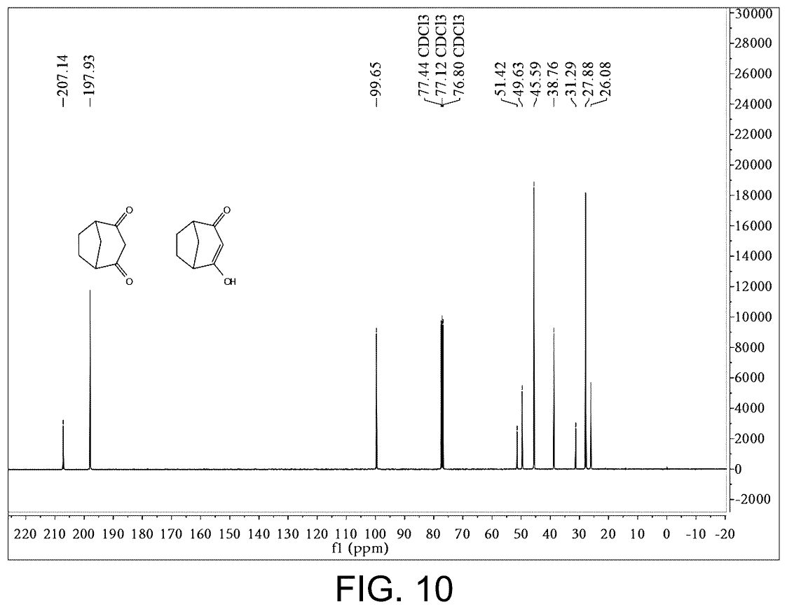

Both keto and enol absorption peaks can be observed in the nuclear magnetic resonance hydrogen spectrum. If this compound is present in keto formula (I), there are only 5 peaks according to a carbon spectrum theory. If this compound is present only in enol formula (Ib), there are only 8 peaks according to the carbon spectrum theory, and the measured results show that the carbon spectrum has 10 peaks, indicating that this compound is in dynamic equilibrium with keto Formula (I) and enol Formula (Ib), and the two configurations simultaneously exist. The nuclear magnetic data are shown in FIG. 9 and FIG. 10.

### 1. Synthesis of 4-methoxybicyclo[3.2.1]-3-octen-2-one

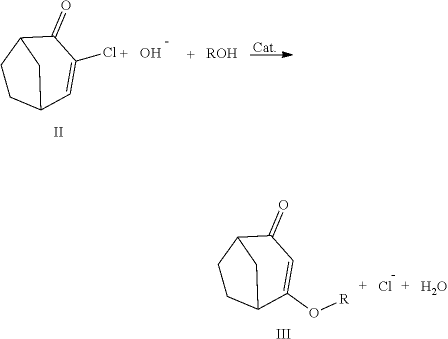

(R=methyl, ethyl, isopropyl, propyl, butyl, etc)

### Example 1A

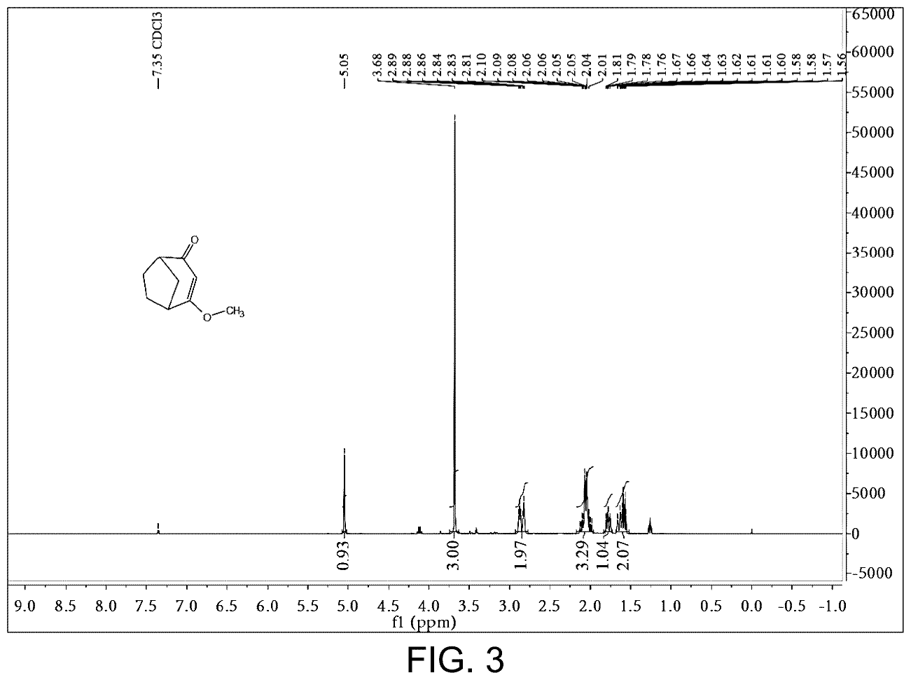

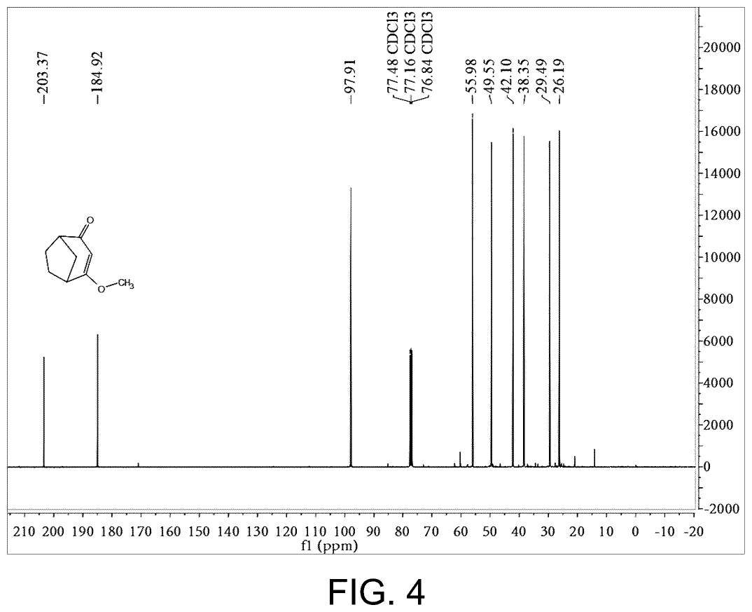

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with methanol (156.0 g, 4.87 mol). Then a sodium hydroxide solid (12.0 g, 0.3 mol) was added thereinto, the obtained mixture was heated and refluxed for reaction, and the reaction stopped when the raw material disappeared. At the end of the reaction, the solvent was removed by distillation and concentration at reduced pressure (−0.095 MPa), and then dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 97.5%, a mass of 15.08 g and a yield of 96.6%. The nuclear magnetic resonance images are shown in FIG. 3 and FIG. 4. The gas phase spectrum of the crude oil product is shown in FIG. 15, showing that the product is mainly the intermediate 4-methoxybicyclo[3.2.1]-3-octen-2-one with fewer impurities and gas phase normalization is greater than 97%.

¹H-NMR (400 MHz, CDCl₃, δ/ppm) 5.05 (t, J=1.4 Hz, 1H), 3.68 (s, 3H), 2.93˜2.77 (m, 2H), 2.17˜1.96 (m, 3H), 1.83˜1.71 (m, 1H), 1.69˜1.52 (m, 2H); ¹³C-NMR (100 MHz, CDCl₃, δ/ppm) 203.37, 184.92, 97.91, 77.48, 77.16, 76.84, 55.98, 49.55, 42.10, 38.35, 29.49, 26.19.

### Example 1B

Under atmospheric pressure, 3-chlorobicyclo[3.2.1]-3-octen-2-one (99%, 15.82 g, 0.1 mol) and a catalyst of sodium cyanide solid (0.98 g, 0.02 mol) were added into a four-necked bottle and dissolved with methanol (156.0 g, 4.87 mol). Then a sodium hydroxide solid (12.0 g, 0.3 mol) was added thereinto, the obtained mixture was heated and refluxed for reaction, and the reaction stopped when the raw material disappeared. At the end of the reaction, the solvent was removed by distillation and concentration at reduced pressure (−0.095 MPa), and then dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 94.5%, a mass of 15.19 g and a yield of 94.3%.

### Example 1C

Under atmospheric pressure, 3-chlorobicyclo[3.2.1]-3-octen-2-one (99%, 15.82 g, 0.1 mol) and a catalyst of sodium cyanide solid (1.47 g, 0.03 mol) were added into a four-necked bottle and dissolved with methanol (156.0 g, 4.87 mol). Then a sodium hydroxide solid (12.0 g, 0.3 mol) was added thereinto, and the obtained mixture was heated and refluxed for reaction, and the reaction stopped when the raw material disappeared. At the end of the reaction, the solvent was removed by distillation and concentration at reduced pressure (−0.095 MPa), and then dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 94.2%, a mass of 14.75 g and a yield of 91.3%.

### Example 2

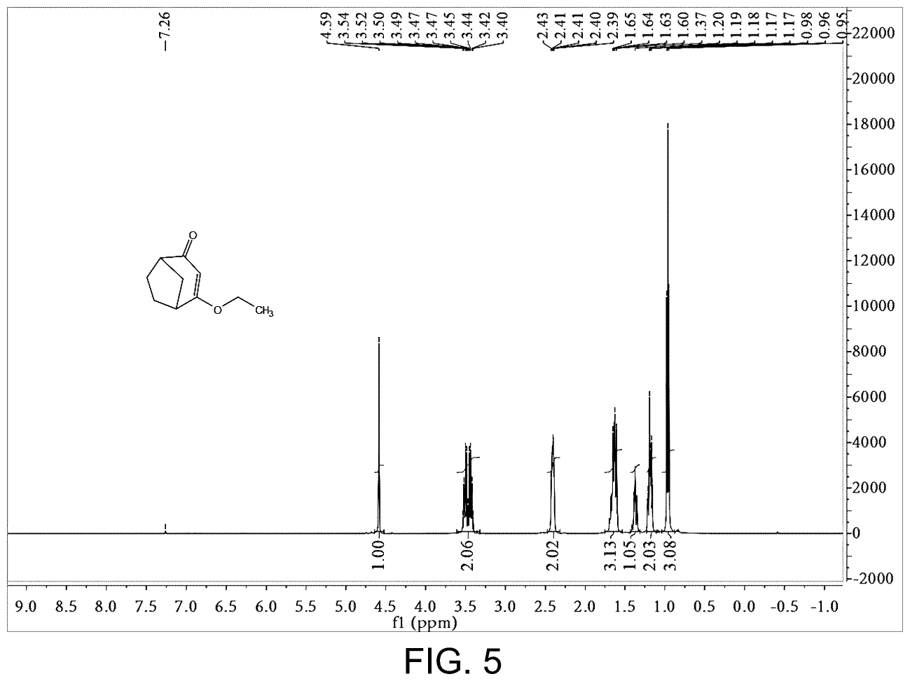

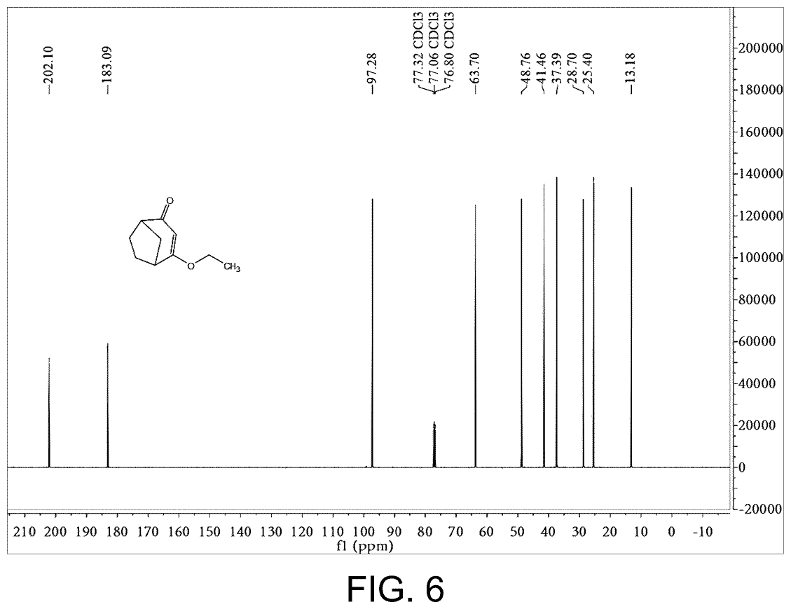

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with ethanol (156.0 g, 3.35 mol). Then a sodium hydroxide solid (12.0 g, 0.3 mol) was added thereinto, the obtained mixture was heated and refluxed for reaction, and the reaction stopped when the raw material disappeared. At the end of the reaction, the solvent was removed by concentration at reduced pressure (−0.095 MPa), and dichloroethane (50.0 g, 0.50 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. The organic phase was distilled under atmospheric pressure to obtain dichloroethane so as to obtain a crude oil product 4-ethoxybicyclo[3.2.1]-3-octen-2-one, with a content of 97.1%, a mass of 16.35 g and a yield of 95.5%. The nuclear magnetic resonance images are shown in FIG. 5 and FIG. 6.

¹H-NMR (500 MHz, CDCl₃, δ/ppm) 4.59 (s, 1H), 3.61˜3.32 (m, 2H), 2.48˜2.32 (m, 2H), 1.75˜1.53 (m, 3H), 1.43˜1.31 (m, 1H), 1.23˜1.11 (m, 2H), 0.96 (t, J=7.1 Hz, 3H). ¹³C-NMR (100 MHz, CDCl₃, δ/ppm) 202.10, 183.09, 97.28, 77.32, 77.06, 76.80, 63.70, 48.76, 41.46, 37.39, 28.70, 25.40, 13.18.

### Example 3

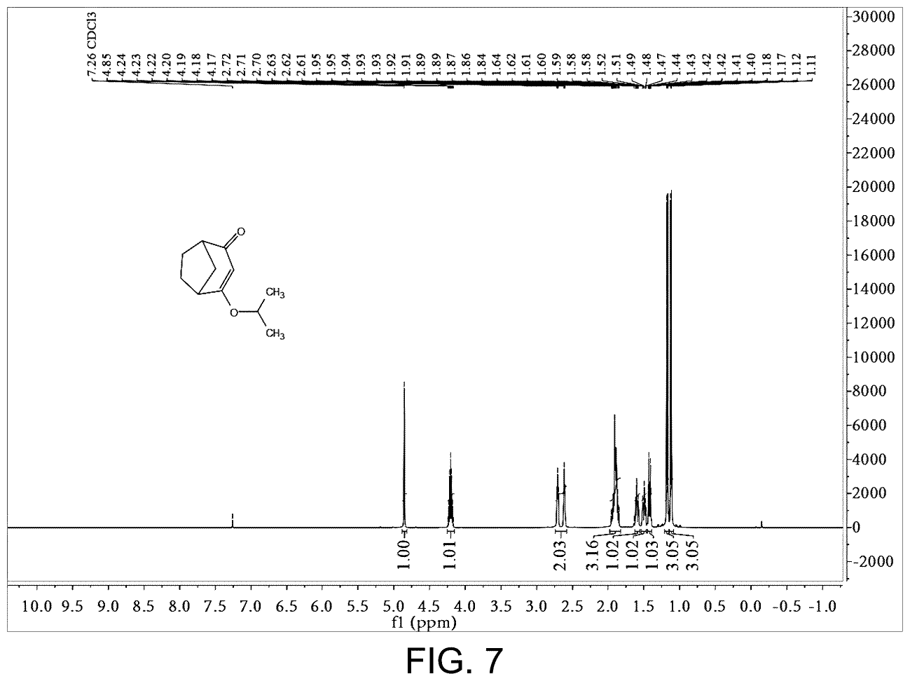

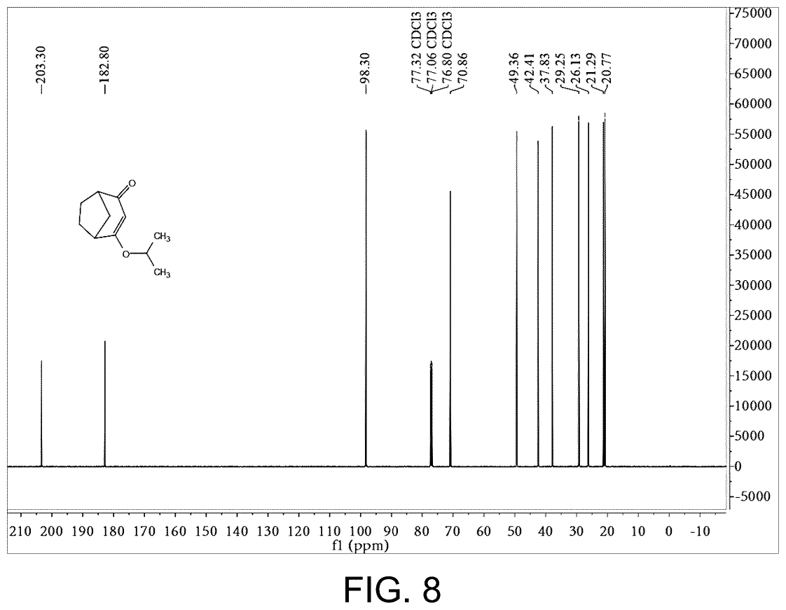

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with isopropanol (156.0 g, 2.57 mol). Then a sodium hydroxide solid (16.0 g, 0.4 mol) was gradually added thereinto, and then the obtained mixture was heated and refluxed for reaction until the raw materials disappeared or no longer reacted. At the end of the reaction, the solvent was removed by concentration at reduced pressure (−0.095 MPa), and dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-isopropoxybicyclo[3.2.1]-3-octen-2-one, with a content of 94.0%, a mass of 17.26 g and a yield of 90.0%. The nuclear magnetic resonance images are shown in FIG. 7 and FIG. 8.

¹H-NMR (500 MHz, CDCl₃, δ/ppm) 4.85 (s, 1H), 4.25˜4.15 (m, 1H), 2.74˜2.58 (m, 2H), 1.98˜1.82 (m, 3H), 1.63˜1.55 (m, 1H), 1.54˜1.46 (m, 1H), 1.44˜1.39 (m, 1H), 1.17 (d, J=6.1 Hz, 3H), 1.12 (d, J=6.1 Hz, 3H). ¹³C-NMR (100 MHz, CDCl₃, δ/ppm) 203.30, 182.80, 98.30, 77.32, 77.06, 76.80, 70.86, 49.36, 42.41, 37.83, 9.25, 26.13, 21.29, 20.77.

### Example 4

Under atmospheric pressure, 3-chlorobicyclo[3.2.1]-3-octen-2-one (99%, 15.82 g, 0.1 mol), a catalyst of sodium cyanide solid (0.245 g, 0.005 mol), ethanol (46.07 g, 1.0 mol) and dichloromethane (156.0 g, 1.82 mol) were added into a four-necked bottle, a sodium hydroxide solid (12.0 g, 0.3 mol) was added thereinto, and then the obtained mixture was heated and refluxed for reaction until the raw materials disappeared or no longer reacted. At the end of the reaction, the solvent was removed by concentration at reduced pressure (−0.095 MPa), and dichloromethane (50.0 g, 0.85 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-ethoxybicyclo[3.2.1]-3-octen-2-one, with a content of 97.3%, a mass of 15.96 g and a yield of 93.4%.

### Example 5

Under atmospheric pressure, a catalyst of sodium cyanide solid (0.245 g, 0.005 mol), a sodium hydroxide solid (12.0 g, 0.3 mol) and methanol (76.0 g, 2.35 mol) were added into a four-necked bottle. 3-chlorobicyclo[3.2.1]-3-octen-2-one (99%, 15.82 g, 0.1 mol) was dissolved with methanol (80.0 g, 2.47 mol), and then the solution of 3-chlorobicyclo[3.2.1]-3-octen-2-one in methanol was dropwise added into the four-necked bottle. The obtained mixture was gradually heated and refluxed for reaction until the raw materials disappeared. At the end of the reaction, the solvent was removed by concentration at reduced pressure (−0.095 MPa), dichloromethane (50.0 g, 0.85 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 97.4%, a mass of 14.77 g and a yield of 94.5%.

### Example 6A

Under atmospheric pressure, 3-chlorobicyclo[3.2.1]-3-octen-2-one (99%, 15.82 g, 0.1 mol) and a catalyst of sodium cyanide solid (0.49 g, 0.01 mol) were added into a four-necked bottle and dissolved with methanol (156.0 g, 4.87 mol). Then a sodium hydroxide solid (12.0 g, 0.3 mol) was added thereinto, and then the reaction was carried out at 30° C. until the conversion of the raw materials stopped. A t the end of the reaction, the solvent was removed by concentration at reduced pressure (−0.095 MPa), and dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 82.6%, a mass of 13.78 g and a yield of 74.8%.

### Example 6B

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with methanol (78.0 g, 2.34 mol). Then a sodium hydroxide solid (12.0 g, 0.3 mol) was gradually added thereinto, then the reaction was carried out at 40° C., the reaction time was prolonged until the remaining raw materials no longer continued to react. At the end of the reaction, the solvent was removed by concentration at reduced pressure (−0.095 MPa), and dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure obtain a crude oil product 4-methoxybicyclo[3.2.1]-3-octen-2-one, with a content of 91.0%, a mass of 15.15 g and a yield of 90.6%.

### Example 6C

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with methanol (46.8 g, 1.46 mol). Then a sodium hydroxide solid (12.0 g, 0.3 mol) was added thereinto, and then the reaction was carried out at 50° C. until the conversion of the raw materials stopped. At the end of the reaction, the solvent was removed by concentration at reduced pressure (−0.095 MPa), and dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure until no fraction was distilled out to obtain a crude oil product 4-methoxybicyclo[3.2.1]-3-octen-2-one, with a content of 92.7%, a mass of 15.05 g and a yield of 91.7%.

### Example 6D

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with methanol (156.0 g, 4.87 mol). Then a sodium hydroxide solid (12.0 g, 0.3 mol) was added thereinto, and then the reaction was carried out at 60° C. until the raw materials disappeared or the conversion of the raw materials stopped. At the end of the reaction, the solvent was removed by concentration at reduced pressure (−0.095 MPa), dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 93.9%, a mass of 14.99 g and a yield of 92.5%.

### Example 7A

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with methanol (78.0 g, 2.43 mol). Then a sodium hydroxide solid (6.0 g, 0.15 mol) was added thereinto, and then the reaction was heated and refluxed for reaction until the conversion of the raw materials stopped. At the end of the reaction, the solvent was removed by concentration at reduced pressure (−0.095 MPa), and dichloromethane (50.0 g, 0.59 mol) and water (50.0 g, 2.78 mol) were added for extraction and liquid separation. A n organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 93.8%, a mass of 14.65 g and a yield of 90.3%.

### Example 7B

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with methanol (156.0 g, 4.87 mol). Then a sodium hydroxide solid (8.0 g, 0.2 mol) was added thereinto, and then the obtained mixture was heated and refluxed for reaction until the conversion of the raw materials stopped. At the end of the reaction, the solvent was removed by concentration at reduced pressure (−0.095 MPa), and dichloromethane (50.0 g, 0.59 mol) and water (50.0 g, 2.78 mol) were added for extraction and liquid separation. An organic phase was subjected to extraction and liquid separation with water (50.0 g, 2.78 mol) again. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 92.6%, a mass of 15.12 g and a yield of 92.0%.

### Example 7C

Under atmospheric pressure, 3-chlorobicyclo[3.2.1]-3-octen-2-one (99%, 15.82 g, 0.1 mol) and a catalyst of sodium cyanide solid (0.245 g, 0.005 mol) were added into a four-necked bottle and dissolved with methanol (234.0 g, 7.31 mol). Then a sodium hydroxide solid (20.0 g, 0.5 mol) was added thereinto, and then the obtained mixture was heated and refluxed for reaction until the conversion of the raw materials stopped. At the end of the reaction, the solvent was removed by concentration at reduced pressure (−0.095 MPa), and dichloromethane (50.0 g, 0.59 mol) and water (50.0 g, 2.78 mol) were added for extraction and liquid separation. The organic phase was washed with water (50.0 g, 2.78 mol) again and then subjected to liquid separation. The cyano radicals in an aqueous phase were quenched separately. Dichloromethane was distilled out from the organic phase under atmospheric pressure to obtain a crude oil product 4-methoxybicyclo[3.2.1]-3-octen-2-one, with a content of 94.8%, a mass of 15.15 g and a yield of 94.4%.

The operation steps of Examples 8-19 are the same as those in Example 1A except that the used raw materials (solvent, alkali, and cyano reagent) are different, and experimental data are shown in Table 1:

TABLE 1
Raw materials and result data of Examples 1-19

| | | | Cyano | | | | |
|---|---|---|---|---|---|---|---|
| | | | reagent | | | | |
| | | | (Use amount | | Molar ratio | | |
| | | | relative to | | of compound | | |
| Number of | Solvent/ | | compound of | Temperature | of Formula | | |
| example | alcohol | Alkali | Formula III) | (° C.) | II to alkali | Purity | Yield |
| 1A | Methanol | Sodium | 0.05eq | Refluxing | 1:3 | 97.5% | 96.6% |
| | | hydroxide | sodium | | | | |
| | | solid | cyanide | | | | |
| | | | (solid) | | | | |
| 1B | Methanol | Sodium | 0.20eq | Refluxing | 1:3 | 94.5% | 94.3% |
| | | hydroxide | sodium | | | | |
| | | solid | cyanide | | | | |
| | | | (solid) | | | | |
| 1C | Methanol | Sodium | 0.30eq | Refluxing | 1:3 | 94.2% | 91.3% |
| | | hydroxide | sodium | | | | |
| | | solid | cyanide | | | | |
| | | | (solid) | | | | |
| 2 | Ethanol | Sodium | 0.05eq | Refluxing | 1:3 | 97.1% | 95.5% |
| | | hydroxide | sodium | | | | |
| | | solid | cyanide | | | | |
| | | | (solid) | | | | |
| 3 | Isopropanol | Sodium | 0.05eq | Refluxing | 1:3 | 94.0% | 90.0% |
| | | hydroxide | sodium | | | | |
| | | solid | cyanide | | | | |
| | | | (solid) | | | | |
| 4 | Dichloromethane, | Sodium | 0.05eq | Refluxing | 1:3 | 97.3% | 93.4% |
| | ethanol | hydroxide | sodium | | | | |
| | | solid | cyanide | | | | |
| | | | (solid) | | | | |
| 5 | Methanol | Sodium | 0.05eq | Refluxing | 1:3 | 97.4% | 94.5% |
| | | hydroxide | sodium | | | | |
| | | solid (reverse | cyanide | | | | |
| | | addition) | (solid) | | | | |
| 6A | Methanol | Sodium | 0.10eq | 30 | 1:3 | 82.6% | 74.8% |
| | | hydroxide | sodium | | | | |
| | | solid | cyanide | | | | |
| | | | (solid) | | | | |
| 6B | Methanol | Sodium | 0.05eq | 40 | 1:3 | 91.0% | 90.6% |
| | | hydroxide | sodium | | | | |
| | | solid | cyanide | | | | |
| | | | (solid) | | | | |
| 6C | Methanol | Sodium | 0.05eq | 50 | 1:3 | 92.7% | 91.7% |
| | | hydroxide | sodium | | | | |
| | | solid | cyanide | | | | |
| | | | (solid) | | | | |
| 6D | Methanol | Sodium | 0.05eq | 60 | 1:3 | 93.9% | 92.5% |
| | | hydroxide | sodium | | | | |
| | | solid | cyanide | | | | |
| | | | (solid) | | | | |
| 7A | Methanol | Sodium | 0.05eq | Refluxing | 1:1.5 | 93.8% | 90.3% |
| | | hydroxide | sodium | | | | |
| | | solid | cyanide | | | | |
| | | | (solid) | | | | |
| 7B | Methanol | Sodium | 0.05eq | Refluxing | 1:2 | 92.6% | 92.0% |
| | | hydroxide | sodium | | | | |
| | | solid | cyanide | | | | |
| | | | (solid) | | | | |
| 7C | Methanol | Sodium | 0.05eq | Refluxing | 1:5 | 94.8% | 94.4% |
| | | hydroxide | sodium | | | | |
| | | solid | cyanide | | | | |
| | | | (solid) | | | | |
| 8 | Methanol | Sodium | 0.05eq | Refluxing | 1:3 | 97.4% | 95.8% |
| | | methoxide | sodium | | | | |
| | | solid | cyanide | | | | |
| | | | (solid) | | | | |
| 9 | Methanol | Sodium | 0.05eq | Refluxing | 1:3 | 97.2% | 91.8% |
| | | ethoxide solid | sodium | | | | |
| | | | cyanide | | | | |
| | | | (solid) | | | | |
| 10 | Ethanol | Sodium | 0.05eq | Refluxing | 1:3 | 97.7% | 90.7% |
| | | ethoxide solid | sodium | | | | |
| | | | cyanide | | | | |
| | | | (solid) | | | | |
| 11 | Ethanol, | Sodium | 0.05eq | Refluxing | 1:3 | 86.6% | 80.9% |
| | toluene | hydroxide | acetone | | | | |
| | | solid | cyanohydrin | | | | |
| 12 | Methanol | Potassium | 0.05eq | Refluxing | 1:3 | 66.5% | 45.5% |
| | | carbonate solid | sodium | | | | |
| | | | cyanide | | | | |
| | | | (solid) | | | | |
| 13 | Methanol | Sodium | 0.05eq | Refluxing | 1:3 | 96.5% | 96.4% |
| | | hydroxide | acetone | | | | |
| | | solid | cyanohydrin | | | | |
| 14 | Methanol | Sodium | 0.05eq | Refluxing | 1:3 | 97.2% | 95.2% |
| | | methoxide | acetone | | | | |
| | | | cyanohydrin | | | | |
| 15 | Methanol | Sodium | 0.05eq | Refluxing | 1:3 | 95.8% | 93.3% |
| | | ethoxide | acetone | | | | |
| | | | cyanohydrin | | | | |
| 16 | Ethanol | Sodium | 0.05eq | Refluxing | 1:3 | 95.1% | 91.7% |
| | | hydroxide | acetone | | | | |
| | | solid | cyanohydrin | | | | |
| 17 | Ethanol | Sodium | 0.05eq | Refluxing | 1:3 | 95.8% | 91.8% |
| | | ethoxide | acetone | | | | |
| | | | cyanohydrin | | | | |
| 18 | Dichloromethane, | Sodium | 0.05eq | Refluxing | 1:3 | 97.1% | 93.6% |
| | ethanol | hydroxide | acetone | | | | |
| | | solid | cyanohydrin | | | | |
| 19 | Methanol | Sodium | 0.05eq | Refluxing | 1:3 | 96.1% | 94.6% |
| | | hydroxide | acetone | | | | |
| | | solid (reverse | cyanohydrin | | | | |
| | | addition) | | | | | |

Note: the reverse addition of the sodium hydroxide solid is to modify a feeding sequence, see Example 5. The feeding sequence is that the sodium hydroxide solid is added, and then 3-chlorobicyclo[3.2.1]-3-octane-2-one is added.

In Examples 1-19, a mixed system of methanol, ethanol, isopropanol, dichloromethane and alcohol can gain a good reaction yield. The reaction yield may be affected when a mixed system of toluene and alcohol is used as a solvent (as shown in Example 11). Strong alkali substances (such as sodium hydroxide and sodium alcohol) are beneficial for the reaction, but the product yield is lower when using weak acid and strong alkali salts with weak alkalinity (as shown in Example 12). Generally speaking, there is no strict requirement for the added amount of the solvent. When the reaction temperature is low, appropriately reducing the amount of methanol and prolonging the reaction time can also obtain products in relatively high yields (for example, in Examples 6B and 6C, when a weight ratio of methanol to the compound of Formula II is 5:1 or less, the product yield is high, while in Example 6, when the reaction temperature is 30° C. and the added amount of methanol is large, products are difficultly obtained in high yields). In Examples 1-5, 6B-10 and 13-19, the yields of the product (4-methoxybicyclo[3.2.1]-3-octen-2-one) can all reach 90% or more. Furthermore, in the above examples, since the amount of the cyano reagent is the amount of the catalyst, cyano radicals are easily separated through extraction and effectively quenched, thereby reducing the risk of hydrogen cyanide production and improving production safety.

The different contents of 4-methoxybicyclo[3.2.1]-3-octane-2-one obtained in the above examples were respectively mixed according to their types, washed with water, dichloromethane-extracted again, and distilled at reduced pressure to remove the solvent, so as to obtain a 4-methoxybicyclo[3.2.1]-3-octane-2-one with a yield of 96%. Then the subsequent hydrolysis step was carried out.

### 2. Acid Hydrolysis Synthesis of bicyclo[3.2.1]-3-octane-2,4-dione

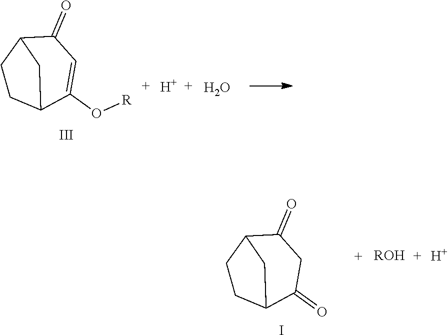

(R=methyl, ethyl, isopropyl, propyl, butyl, etc)

### Example 20

Under atmospheric pressure, 4-methoxybicyclo[3.2.1]-3-octen-2-one (96%, 15.85 g, 0.1 mol) was added into a four-necked bottle, water (50.0 g, 2.78 mol) and a 20% sulfuric acid aqueous solution (49.0 g, 0.1 mol) were added thereinto. The obtained mixture was slowly heated to a refluxing temperature to gradually evaporate out methanol. After the raw materials disappeared and the reaction ended, the temperature was reduced to room temperature. Dichloromethane (100.0 g, 1.17 mol) was added for extraction and liquid separation, and dichloromethane was distilled out from an organic phase under atmospheric pressure to obtain a light yellow solid product bicyclo[3.2.1]-3-octane-2,4-dione. The solid product was washed with water (50.0 g, 2.78 mol), filtered at reduced pressure to obtain a wet product, and then the wet product was dried to obtain 13.72 g of a product, with a content of 98.9% and a yield of 98.2%. The nuclear magnetic resonance images are shown in FIG. 9 and FIG. 10. The gas phase spectrum of the product is shown in FIG. 16, showing that there are almost no visible impurities except for the residual raw material 3-chlorobicyclo[3.2.1]-3-octen-2-one in the previous step, and the gas phase normalization is greater than 98%.

¹H-NMR: (600 MHz, CDCl₃, δ/ppm)

Enol formula: 10.88 (s, 1H), 5.14 (s, 1H), 2.89 (s, 2H), 2.12˜2.05 (m, 2H), 1.97˜1.90 (m, 2H), 1.78˜1.66 (m, 2H). Dione formula: 3.37˜3.11 (m, 2H), 3.03 (s, 2H), 2.19˜2.12 (m, 2H), 2.06˜1.98 (m, 2H), 1.61˜1.51 (m, 2H).

### Example 21

Under atmospheric pressure, 4-methoxybicyclo[3.2.1]-3-octen-2-one (96%, 15.85 g, 0.1 mol) was added into a four-necked bottle, and a 20% sulfuric acid aqueous solution (147.0 g, 0.3 mol) was added thereinto, and the obtained mixture was heated to a refluxing temperature to gradually evaporate out methanol. After the raw materials disappeared and the reaction ended, the temperature was reduced to room temperature. Dichloromethane (100.0 g, 1.17 mol) was added for extraction and liquid separation, and dichloromethane was distilled out from an organic phase under atmospheric pressure. A light yellow solid product bicyclo[3.2.1]-3-octane-2,4-dione was obtained, and the solid product was leached with water (50.0 g, 2.78 mol) and filtered at reduced pressure to obtain a wet product. And the wet product was dried to obtain a product with a content of 93.2%, a mass of 13.74 g, and a yield of 92.7%.

### Example 22

Under atmospheric pressure, 4-methoxybicyclo[3.2.1]-3-octen-2-one (96%, 15.85 g, 0.1 mol) was added into a four-necked bottle. Water (50.0 g, 2.78 mol) and a 20% sulfuric acid aqueous solution (27.0 g, 0.05 mol) were added thereinto, the obtained mixture was heated to a refluxing temperature to gradually evaporate out methanol. After the raw materials disappeared and the reaction ended, the temperature was reduced to room temperature. Dichloromethane (100.0 g, 1.17 mol) was added for extraction and liquid separation, and dichloromethane was distilled out from an organic phase under atmospheric pressure. A light yellow solid product bicyclo[3.2.1]-3-octane-2,4-dione was obtained, and the solid product was leached with water (50.0 g, 2.78 mol) and filtered at reduced pressure to obtain a wet product. And the wet product was dried to obtain a product with a content of 93.6%, a mass of 13.57 g, and a yield of 91.9%.

### Example 23

Under atmospheric pressure, 4-methoxybicyclo[3.2.1]-3-octen-2-one (96%, 15.85 g, 0.1 mol) was added into a four-necked bottle, and a 20% sulfuric acid aqueous solution (98.0 g, 0.2 mol) was added thereinto. The obtained mixture was heated to a refluxing temperature to gradually evaporate out methanol. After the reaction ended, the temperature was reduced to room temperature. Dichloromethane (100.0 g, 1.17 mol) was added for extraction and liquid separation, and dichloromethane was distilled out from an organic phase under atmospheric pressure. A light yellow solid product bicyclo[3.2.1]-3-octane-2,4-dione was obtained, and the solid product was leached with water (50.0 g, 2.78 mol) and filtered at reduced pressure to obtain a wet product. And the wet product was dried to obtain a product with a content of 95.2%, a mass of 13.72 g, and a yield of 94.5%.

### Example 24

Under atmospheric pressure, 4-methoxybicyclo[3.2.1]-3-octen-2-one (96%, 15.85 g, 0.1 mol) was added into a four-necked bottle, and water (50.0 g, 2.78 mol) and a 20% sulfuric acid aqueous solution (49.0 g, 0.1 mol) were added thereinto. The obtained mixture was heated to 50° C. After the reaction ended, the temperature was reduced to room temperature. Dichloromethane (100.0 g, 1.17 mol) was added for extraction and liquid separation, and dichloromethane was distilled out from an organic phase under atmospheric pressure. A light yellow solid product bicyclo[3.2.1]-3-octane-2,4-dione was obtained, and the solid product was leached with water (50.0 g, 2.78 mol) and filtered at reduced pressure to obtain a wet product. And the wet product was dried to obtain a product with a content of 93.2%, a mass of 13.76 g, and a yield of 92.8%.

### Example 24B

Under atmospheric pressure, 4-methoxybicyclo[3.2.1]-3-octen-2-one (96%, 15.85 g, 0.1 mol) was added into a four-necked bottle, and water (50.0 g, 2.78 mol) and a 20% sulfuric acid aqueous solution (49.0 g, 0.1 mol) were added thereinto. The obtained mixture was heated to 60° C. After the reaction ended, the temperature was reduced to room temperature. Dichloromethane (100.0 g, 1.17 mol) was added for extraction and liquid separation, and dichloromethane was distilled out from an organic phase under atmospheric pressure. A light yellow solid product bicyclo[3.2.1]-3-octane-2,4-dione was obtained, and the solid product was leached with water (50.0 g, 2.78 mol) and filtered at reduced pressure to obtain a wet product. And the wet product was dried to obtain a product with a content of 94.2%, a mass of 13.68 g, and a yield of 93.3%.

### Example 24C

Under atmospheric pressure, 4-methoxybicyclo[3.2.1]-3-octen-2-one (96%, 15.85 g, 0.1 mol) was added into a four-necked bottle, and water (50.0 g, 2.78 mol) and a 20% sulfuric acid aqueous solution (49.0 g, 0.1 mol) were added thereinto. And the obtained mixture was heated to 80° C. After the reaction ended, the temperature was reduced to room temperature. Dichloromethane (100.0 g, 1.17 mol) was added for extraction and liquid separation, and dichloromethane was distilled out from an organic phase under atmospheric pressure. A light yellow solid product bicyclo[3.2.1]-3-octane-2,4-dione was obtained, and the solid product was leached with water (50.0 g, 2.78 mol) and filtered at reduced pressure to obtain a wet product. And the wet product was dried to obtain a product with a content of 95.7%, a mass of 13.90 g, and a yield of 96.3%.

The reaction operations of acid hydrolysis in Examples 25-26 are the same as those in Example 20 except that acids are different, and experimental data are shown in Table 2:

TABLE 2
Acid hydrolysis data of examples 20-26

| Number of | | | Molar ratio of raw | Temperature | | |
|---|---|---|---|---|---|---|
| example | Solvent | Acid | material to acid | (° C.) | Content | Yield |
| 20 | Water | Sulfuric acid | 1:1 | Refluxing | 98.9% | 98.2% |
| 21 | Water | Sulfuric acid | 1:3 | Refluxing | 93.2% | 92.7% |
| 22 | Water | Sulfuric acid | 1:0.5 | Refluxing | 93.6% | 91.9% |
| 23 | Water | Sulfuric acid | 1:2 | Refluxing | 95.2% | 94.5% |
| 24 | Water | Sulfuric acid | 1:1 | 50° C. | 93.2% | 92.8% |
| 24B | Water | Sulfuric acid | 1:1 | 60° C. | 94.2% | 93.3% |
| 24C | Water | Sulfuric acid | 1:1 | 80° C. | 95.7% | 96.3% |
| 25 | Water | P-toluene | 1:1 | Refluxing | 96.1% | 96.0% |
| | | sulfonic acid | | | | |
| 26 | Water | Trifuloroacetic | 1:1 | Refluxing | 96.7% | 96.2% |
| | | acid | | | | |

### 3. Alkali Hydrolysis Synthesis of bicyclo[3.2.1]-3-octane-2,4-dione

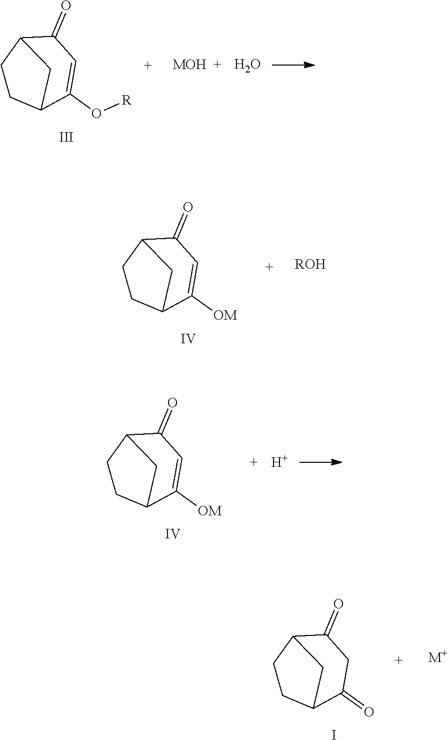

(R=methyl, ethyl, isopropyl, propyl, butyl, etc. M=Na, K, Li, Mg, etc.)

### Example 27

Under atmospheric pressure, 4-methoxybicyclo[3.2.1]-3-octen-2-one (96%, 15.85 g, 0.1 mol) was added with water (50.0 g, 2.78 mol) and a 50% sodium hydroxide aqueous solution (24.0 g, 0.3 mol), and the above mixed material was slowly heated to a refluxing temperature to gradually evaporate out methanol. After the raw materials completely disappeared, the reaction stopped and the temperature was reduced to room temperature. Hydrochloric acid was added to adjust the above reaction solution to pH=1-3 to precipitate out a solid, and the solid was filtered at reduced pressure and leached with water (50.0 g, 2.78 mol) to obtain a wet product. The wet product was dried to obtain a light yellow product bicyclo[3.2.1]-3-octane-2,4-dione, with a content of 98.8%, a mass of 13.66 g and a yield of 97.7%.

### Example 28A

Under atmospheric pressure, 4-methoxybicyclo[3.2.1]-3-octen-2-one (96%, 15.85 g, 0.1 mol) was added with water (50.0 g, 2.78 mol) and a 50% sodium hydroxide aqueous solution (24.0 g, 0.3 mol), and the above mixed materials started to react at 50° C. The above reaction product was gradually heated after the raw materials no longer reacted, so as to evaporate out methanol, and then the temperature was reduced to room temperature after no fraction was distilled out. Hydrochloric acid was added to adjust the above reaction solution to pH=1-3 to precipitate out a solid, and the solid was filtered at reduced pressure and leached with water (50.0 g, 2.78 mol) to obtain a wet product. The wet product was dried to obtain a light yellow product bicyclo[3.2.1]-3-octane-2,4-dione, with a content of 93.2%, a mass of 13.74 g and a yield of 92.7%.

### Example 28B

Under atmospheric pressure, 4-methoxybicyclo[3.2.1]-3-octen-2-one (96%, 15.85 g, 0.1 mol) was added with water (50.0 g, 2.78 mol) and a 50% sodium hydroxide aqueous solution (24.0 g, 0.3 mol), and the above mixed materials started to react at 70° C. The above reaction product was gradually heated after the raw materials no longer reacted, so as to evaporate out methanol, and then the temperature was reduced to room temperature after no fraction was distilled out. Hydrochloric acid was added to adjust the above reaction solution to pH=1-3 to precipitate out a solid, and the solid was filtered at reduced pressure and leached with water (50.0 g, 2.78 mol) to obtain a wet product. The wet product was dried to obtain a light yellow product bicyclo[3.2.1]-3-octane-2,4-dione, with a content of 93.4%, a mass of 13.76 g and a yield of 93.0%.

### Example 28C

Under atmospheric pressure, 4-methoxybicyclo[3.2.1]-3-octen-2-one (96%, 15.85 g, 0.1 mol) was added with water (50.0 g, 2.78 mol) and a 50% sodium hydroxide aqueous solution (24.0 g, 0.3 mol), and the reaction started at 80° C., and then methanol was evaporated after the raw materials completely disappeared. The temperature was reduced to room temperature after no fraction was distilled out. Hydrochloric acid was added to adjust the above reaction solution to pH=1-3 to precipitate out a solid, and the solid was filtered at reduced pressure and leached with water (50.0 g, 2.78 mol) to obtain a wet product. The wet product was dried to obtain a light yellow product bicyclo[3.2.1]-3-octane-2,4-dione, with a content of 96.1%, a mass of 13.57 g and a yield of 94.4%.

### Example 29A

Under atmospheric pressure, 4-methoxybicyclo[3.2.1]-3-octen-2-one (96%, 15.85 g, 0.1 mol) was added with water (50.0 g, 2.78 mol) and a 50% sodium hydroxide aqueous solution (8.0 g, 0.1 mol), and the above mixed material was slowly heated to a refluxing temperature to gradually evaporate out methanol, and the temperature was reduced to room temperature after methanol was no longer evaporated at the end of the reaction. Hydrochloric acid was added to adjust the above reaction solution to pH=1-3 to precipitate out a solid, and the solid was filtered at reduced pressure and leached with water (50.0 g, 2.78 mol) to obtain a wet product. The wet product was dried to obtain a light yellow product bicyclo[3.2.1]-3-octane-2,4-dione, with a content of 93.1%, a mass of 13.71 g and a yield of 92.4%.

### Example 29B

Under atmospheric pressure, 4-methoxybicyclo[3.2.1]-3-octen-2-one (96%, 15.85 g, 0.1 mol) was added with water (50.0 g, 2.78 mol) and a 50% sodium hydroxide aqueous solution (16.0 g, 0.2 mol), and the above mixed material was slowly heated to a refluxing temperature to gradually evaporate out methanol, and then the temperature was reduced to room temperature after the reaction ended. Hydrochloric acid was added to adjust the above reaction solution to pH=1-3 to precipitate out a solid, and the solid was filtered at reduced pressure and leached with water (50.0 g, 2.78 mol) to obtain a wet product. The wet product was dried to obtain a light yellow product bicyclo[3.2.1]-3-octane-2,4-dione, with a content of 93.4%, a mass of 13.80 g and a yield of 93.3%.

### Example 29C

Under atmospheric pressure, 4-methoxybicyclo[3.2.1]-3-octen-2-one (96%, 15.85 g, 0.1 mol) was added with water (50.0 g, 2.78 mol) and a 50% sodium hydroxide aqueous solution (40.0 g, 0.5 mol), and the above mixed material was slowly heated to a refluxing temperature to gradually evaporate out methanol, and then the temperature was reduced to room temperature after the reaction ended. Hydrochloric acid was added to adjust the above reaction solution to pH=1-3 to precipitate out a solid, and the solid was filtered at reduced pressure and leached with water (50.0 g, 2.78 mol) to obtain a wet product. The wet product was dried to obtain a light yellow product bicyclo[3.2.1]-3-octane-2,4-dione, with a content of 94.2%, a mass of 13.71 g and a yield of 93.5%.

The operations of alkali hydrolysis in Examples 3-32 are the same as those in Example 27 except that materials are different, and experimental data are shown in Table 3 below:

TABLE 3
Alkali hydrolysis experimental data in Examples 27-32

| | | | | | Molar ratio | | |
|---|---|---|---|---|---|---|---|
| Number of | | | Acid | Temperature | of raw | | |
| example | Solvent | Alkali | adjustment | (° C.) | material:alkali | Content | Yield |
| 27 | Water | Sodium | Hydrochloric | Refluxing | 1:3 | 98.8% | 97.7% |
| | | hydroxide | acid | | | | |
| | | solution | | | | | |
| 28A | Water | Sodium | Hydrochloric | 50 | 1:3 | 93.2% | 92.7% |
| | | hydroxide | acid | | | | |
| | | solution | | | | | |
| 28B | Water | Sodium | Hydrochloric | 60 | 1:3 | 93.4% | 93.0% |
| | | hydroxide | acid | | | | |
| | | solution | | | | | |
| 28C | Water | Sodium | Hydrochloric | 80 | 1:3 | 96.1% | 94.4% |
| | | hydroxide | acid | | | | |
| | | solution | | | | | |
| 29A | Water | Sodium | Hydrochloric | Refluxing | 1:1 | 93.1% | 92.4% |
| | | hydroxide | acid | | | | |
| | | solution | | | | | |
| 29B | Water | Sodium | Hydrochloric | Refluxing | 1:2 | 93.4% | 93.3% |
| | | hydroxide | acid | | | | |
| | | solution | | | | | |
| 29C | Water | Sodium | Hydrochloric | Refluxing | 1:5 | 94.2% | 93.5% |
| | | hydroxide | acid | | | | |
| | | solution | | | | | |
| 30 | Water | Sodium | Diluted | Refluxing | 1:3 | 98.2% | 97.4% |
| | | hydroxide | sulfuric acid | | | | |
| | | solution | | | | | |
| 31 | Water | Sodium | Hydrochloric | Refluxing | 1:3 | 95.2% | 94.6% |
| | | methoxide | acid | | | | |
| | | solution | | | | | |
| 32 | Water | Sodium | Hydrochloric | Refluxing | 1:3 | 94.8% | 94.4% |
| | | ethoxide | acid | | | | |
| | | solution | | | | | |

Examples 1-32 were carried out by a two-step method—that is, 3-chlorobicyclo[3.2.1]-3-octen-2-one reacted with an alkali and an alcohol under an anhydrous condition in the presence of a cyanide catalyst, and then cyano radicals were removed through extraction; then the product was subjected to hydrolysis—acid adjustment after alkali hydrolysis, or acid hydrolysis to obtain the target product (bicyclo[3.2.1]-3-octane-2,4-dione). The above two-step method not only ensures the yield, but also can effectively remove the production hazards caused by hydrogen cyanide, and can remove byproducts from the reaction process. The product content is high, which is conducive to subsequent reactions.

### 4. One-Pot Synthesis Method of bicyclo[3.2.1]-3-octane-2,4-dione

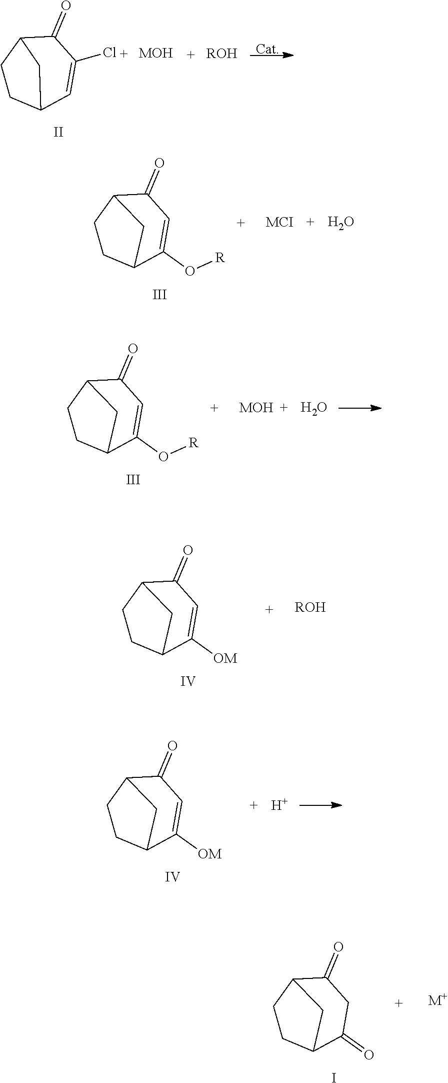

(R=methyl, ethyl, isopropyl, propyl, butyl, etc. M=Na, K, Li, Mg, etc.)

### Example 33A

Under atmospheric pressure, 3-chlorobicyclo[3.2.1]-3-octen-2-one (99%, 15.85 g, 0.1 mol) and a catalyst of acetone cyanohydrin (0.42 g, 0.005 mol) were added into a four-necked bottle and dissolved with methanol (156.0 g, 2.57 mol). A sodium hydroxide solid (12.0 g, 0.3 mol) was added thereinto, and then the obtained mixture was heated until refluxing for reaction. The solvent was distilled out after the reaction was carried out until the raw materials disappeared or no longer reacted. After no solvent was distilled out, water (158.0 g, 8.77 mol) was added and the mixture was continued to be heated until refluxing for reaction, and the temperature was reduced to room temperature after 4-methoxybicyclo[3.2.1]-3-octen-2-one completely disappeared. Hydrochloric acid was added to adjust the above solution to pH=1-3 so that a solid was precipitated out, and hydrocyanic acid appearing during acid adjustment was absorbed at reduced pressured through a safety absorption bottle. The reaction system was filtered at reduced pressure, leached with water (50.0 g, 2.78 mol), and filtered at reduced pressure to obtain a wet product. The wet product was dried to obtain a light yellow product bicyclo[3.2.1]-3-octane-2,4-dione, with a content of 97.2%, a mass of 13.66 g and a yield of 96.1%. The gas phase spectrum of the light yellow solid product is shown in FIG. 18, showing that the target product is bicyclo[3.2.1]-3-octane-2,4-dione at 12.43 min, with almost no impurity peaks and a normalized purity of 97.2%.

The operation steps in Examples 34-37 are the same as those in Example 33A except that the materials are different, and experimental data are shown in Table 4 below:

TABLE 4
Experimental data of one-pot method in each of Examples 33-37

| | | | Cyano reagent | | | | |
|---|---|---|---|---|---|---|---|
| | | | (relative to | | | | |
| | | | amount of | | | | |
| Number of | | | compound of | Adjustment | | | |
| example | Solvent | Alkali | Formula II) | of pH | Content | Yield | Note |
| 33A | Methanol | Sodium | 5% acetone | Hydrochloric | 97.2% | 96.1% | |
| | | hydroxide | cyanohydrin | acid | | | |
| | | solid | | | | | |
| 33B | Methanol | Sodium | 20% acetone | Hydrochloric | 94.4% | 93.0% | Acetone |
| | | hydroxide | cyanohydrin | acid | | | cyanohydrin |
| | | solid | | | | | is 0.2eq |
| 33C | Methanol | Sodium | 30% acetone | Hydrochloric | 92.3% | 89.8% | Acetone |
| | | hydroxide | cyanohydrin | acid | | | cyanohydrin |
| | | solid | | | | | is 0.3eq |
| 33D | Methanol | Sodium | 5% acetone | Hydrochloric | 83.4% | 78.5% | Compound |
| | | hydroxide | cyanohydrin | acid | | | of formula |
| | | solid | | | | | II:alkali = 1:1.5 |
| 33E | Methanol | Sodium | 5% acetone | Hydrochloric | 94.6% | 93.5% | Compound |
| | | hydroxide | cyanohydrin | acid | | | of formula |
| | | solid | | | | | II:alkali = 1:2, |
| | | | | | | | the amount |
| | | | | | | | of solvent |
| | | | | | | | methano is |
| | | | | | | | reduced |
| 33F | Methanol | Sodium | 5% acetone | Hydrochloric | 92.3% | 89.9% | Compound |
| | | hydroxide | cyanohydrin | acid | | | of formula |
| | | solid | | | | | II:alkali = 1:5 |
| 33G | Methanol | Sodium | Sodium | Hydrochloric | 94.1% | 91.0% | The reaction |
| | | hydroxide | cyanide (solid) | acid | | | temperature |
| | | solid | | | | | in the |
| | | | | | | | previous |
| | | | | | | | stage is 40° C., |
| | | | | | | | and the |
| | | | | | | | amount of |
| | | | | | | | solvent |
| | | | | | | | methanol is |
| | | | | | | | reduced |
| 33H | Methanol | Sodium | Sodium | Hydrochloric | 94.5% | 91.9% | The reaction |
| | | hydroxide | cyanide (solid) | acid | | | temperature |
| | | solid | | | | | in the |
| | | | | | | | previous |
| | | | | | | | stage is 60° C. |
| 33I | Methanol | Sodium | Sodium | Hydrochloric | 94.1% | 92.2% | The reaction |
| | | hydroxide | cyanide (solid) | acid | | | temperature |
| | | solid | | | | | in the later |
| | | | | | | | stage is 50° C., |
| | | | | | | | the amount of |
| | | | | | | | water in the |
| | | | | | | | later stage is |
| | | | | | | | reduced |
| 33J | Methanol | Sodium | Sodium | Hydrochloric | 95.6% | 93.7% | The reaction |
| | | hydroxide | cyanide (solid) | acid | | | temperature |
| | | solid | | | | | in the later |
| | | | | | | | stage is 70° C. |
| 34 | Methanol | Sodium | Sodium | Hydrochloric | 94.5% | 93.2% | |
| | | methoxide | cyanide (solid) | acid | | | |
| 35 | Dichloromethane | Sodium | Acetone | Hydrochloric | 95.4% | 95.2% | |
| | and | hydroxide | cyanohydrin | acid | | | |
| | methanol | solid | | | | | |
| 36 | Ethanol | Sodium | Sodium | Hydrochloric | 95.5% | 93.1% | The amount |
| | | ethoxide | cyanide (solid) | acid | | | of solvent |
| | | | | | | | ethanol is |
| | | | | | | | reduced |
| 37 | Isopropanol | Sodium | Sodium | Hydrochloric | 93.5% | 92.2% | The amount |
| | | hydroxide | cyanide (solid) | acid | | | of solvent |
| | | solid | | | | | isopropanol |
| | | | | | | | is reduced |

The molar ratio of the compound of Formula II to the alkali in Examples 33A-33C and 33G-37 is 1:3.

In Examples 33A-37, preparation was carried out using a one-pot method—that is, 3-chlorobicyclo[3.2.1]-3-octen-2-one reacted with an alkali and an alcohol under an anhydrous condition in the presence of a cyanide catalyst, then the solvent was evaporated out, water was supplemented to continue the reaction, and then acid adjustment was carried out to obtain the target product (bicyclo[3.2.1]-3-octane-2,4-dione). In the above one-pot method, two reactions were carried out successively. The first reaction was carried out under an anhydrous condition, and the second hydrolysis reaction had no side reaction competition. The reaction yield can be greatly improved, for example, in Example 33A, the yield can reach over 96%, and the product purity can reach 97%, which can be directly used for subsequent reactions.

Compared with the two-step method, the one-pot method in the present application can achieve continuous reaction and relatively simple treatment, so as to reduce separation losses and improve reaction yield. However, there are small amounts of cyanide compounds in the reaction system, resulting in low product content and the presence of some byproducts. The advantage of the two-step method is that the byproducts generated in each step of the reaction can be removed, resulting in a decrease in yield, but the product content is high, which is beneficial for subsequent reactions.

### Comparative Example 1 (Scheme A of Process Steps d) and e) of the Step-by-Step Method in CN1440376A)

1) Preparation of 4-cyanobicyclo[3.2.1]-3-octen-2-one: a mixture of 3-chlorobicyclo[3.2.1]oct-3-en-2-one (0.5 g, 0.032 mmol), triethylamine (0.92 g, 0.032 mmol), acetone cyanohydrin (0.27 g, 0.32 mmol) and methanol (5 mL) were stirred for 24 hours at room temperature, then poured into water and extracted with ethyl acetate. A n organic phase was washed with 2N hydrochloric acid, dried with sodium sulfate, and concentrated to obtain a product.

By repeating the synthesis method of this route, the influence of different temperatures on the reaction results was further explored. The reaction process is detected through a gas phase. When the raw materials are left and the reaction no longer continues or the raw materials completely disappear, the reaction is terminated and post-treatment is carried out. Experimental data are shown in Table 5.

TABLE 5
Comparison data of cyano reactions of 3-chlorobicyclo[3.2.1]-3-
octen-2-one at different temperatures

| Serial | | | Cyano | Temperature | Yield of | |
|---|---|---|---|---|---|---|
| number | Solvent | Alkali | reagent | (° C.) | formula II | Note |
| Original | Methanol | Triethylamide | Acetone | 25 | 85% | Original data of |
| record | | | cyanohydrin | | | Example A of step d |
| data | | | | | | of CN1440376A |
| 1 | Methanol | triethylamide | Acetone | 25 | 80.1% | Data obtained by |
| | | | cyanohydrin | | | repeating |
| 2 | Methanol | Triethylamide | Acetone | 25 | 80.5% | experiment of |
| | | | cyanohydrin | | | CN1440376A by the |
| 3 | Methanol | Triethylamide | Acetone | 25 | 81.2% | inventors |
| | | | cyanohydrin | | | |
| 4 | Methanol | Triethylamide | Acetone | 10 | 77.2% | Data obtained by |
| | | | cyanohydrin | | | optimizing |
| 5 | Methanol | Triethylamide | Acetone | 30 | 81.3% | experiment of |
| | | | cyanohydrin | | | CN1440376A by the |
| 6 | Methanol | Triethylamide | Acetone | 50 | 74.6% | inventors |
| | | | cyanohydrin | | | |

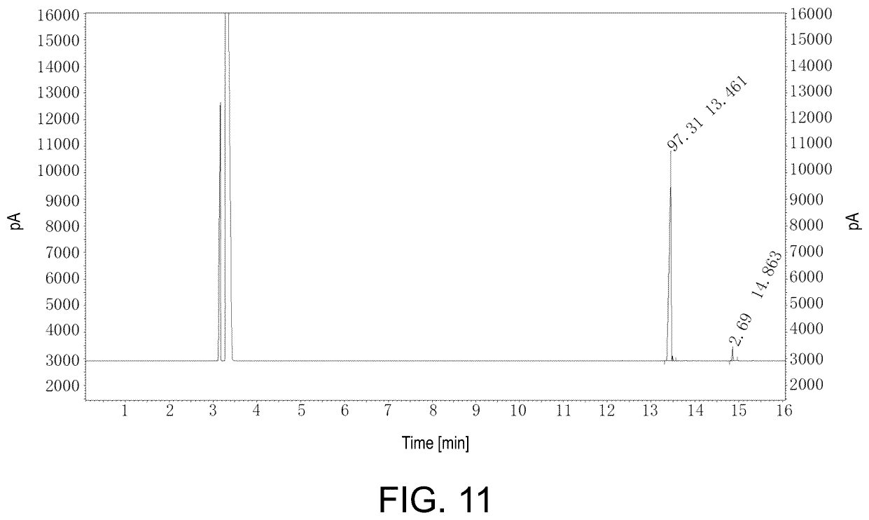

The inventors repeated experiments many times, but it was difficult to improve the yield of Formula II. The inventors speculated that due to competition caused by multiple side reactions, the materials obtained from the initial and completed reactions of the reaction route (obtained according to the method in step 1) were extracted, washed with hydrochloric acid, and dried with sodium sulfate to obtain purified materials. The gas chromatograms of the purified materials obtained from the initial and completed reactions are shown in FIG. 11 and FIG. 12, respectively. From FIG. 11, it can be seen that after the material at the beginning of the reaction is purified, only one impurity (14.86 min) is present, while FIG. 12 after the reaction is completed shows that the purified material still has many impurities, wherein 4-cyanobicyclo[3.2.1]-3-octen-2-one intermediate is generated at 13.51 min.

In the steps of this comparative example, the amount of acetone cyanohydrin is equivalent to the amount of 3-chlorobicyclo[3.2.1]-3-octen-2-one in order to generate the 4-cyanobicyclo[3.2.1]-3-octen-2-one intermediate. In this step, there is a small amount of unreacted acetone cyanohydrin that is soluble in organic solvents and difficult to extract and separate, so there is a risk of generating hydrogen cyanide in organic phase acid adjustment.

2) Preparation of bicyclo[3.2.1]-3-octane-2,4-dione: 4-cyanobicyclo[3.2.1]-3-octen-2-one (0.02 g, 0.14 mmol) in step 1 was treated with a potassium hydroxide aqueous solution (0.5%, 20 mol), and stirred for 2 hours at room temperature. The mixture was acidified with hydrochloric acid and extracted with ethyl acetate, and the organic phase was dried and concentrated with sodium sulfate.

When the inventors of the present application repeated the synthesis method of this step, the reaction process was detected by gas phase. When the remaining reaction of the raw materials no longer continued or the raw materials completely disappeared, the reaction was terminated and post-treatment was carried out. Experimental data are shown in Table 6 below.

TABLE 6
Comparison data of alkali hydrolysis reaction of 4-cyanobicyclo[3.2.1]-3-octen-2-one

| Serial | Temperature | | | | |
|---|---|---|---|---|---|
| number | (° C.) | Alkali | Acid | Formula V | Note |
| Original | 25 | 0.5% sodium | Hydrochloric | 36% | Original data of |
| data | | hydroxide solution | acid | | Example A of step e |
| | | | | | of CN 1440376A |
| 7 | 25 | 0.5% sodium | Hydrochloric | 35.8% | Data obtained by |
| | | hydroxide solution | acid | | repeating |
| 8 | 25 | 0.5% sodium | Hydrochloric | 36.6% | experiment of |
| | | hydroxide solution | acid | | CN 1440376A by |
| 9 | 25 | 0.5% sodium | Hydrochloric | 36.5% | the inventors |
| | | hydroxide solution | acid | | |

The yield of the hydrolysis step in this step was relatively low, and the inventors speculated that it may be due to the competition caused by multiple side reactions and the generation of more byproducts, resulting in a lower yield.

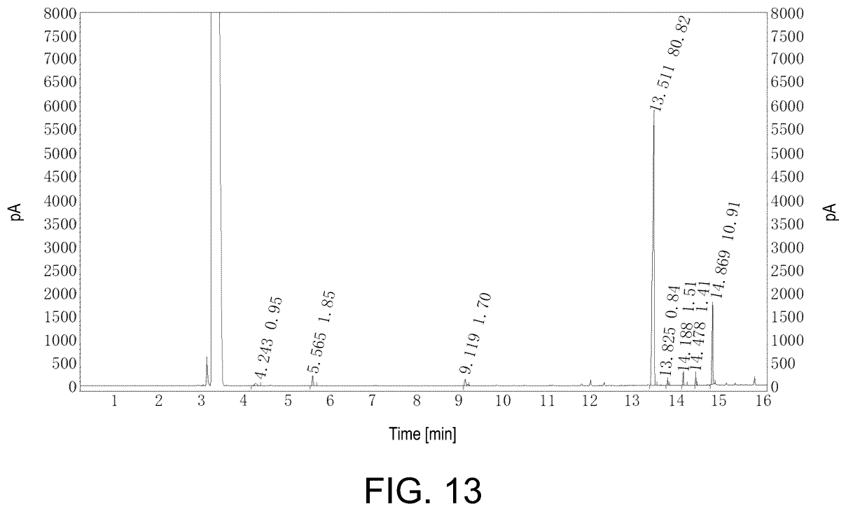

The inventors repeated experiments many times, but it was difficult to improve the hydrolysis yield. The inventors speculated that due to the competition caused by multiple side reactions, the materials from step 2) of the reaction route after hydrolysis reaction for 1 hour and the materials after hydrolysis reaction completion were taken, and acidified, extracted, and dried with sodium sulfate to obtain purified materials. The gas chromatograms of the purified materials after hydrolysis reaction for 1 hour and the purified materials after reaction completion are shown in FIG. 13 and FIG. 14, respectively. From FIG. 13, it can be seen that after the material at the beginning of the reaction is purified, only one impurity (14.86 min) is present, while FIG. 14 after the reaction is completed shows that at 13.51 min, the 4-cyanobicyclo[3.2.1]-3-octen-2-one intermediate basically disappeared after hydrolysis, and other impurity peaks increased before and after the reaction, indicating severe competition among byproducts.

In step 2) of this comparative example, the 4-cyanobicyclo[3.2.1]-3-octen-2-one intermediate can release cyano radicals during the hydrolysis, and a large amount of highly toxic hydrogen cyanide gas can be generated during the acid adjustment, thereby posing a significant safety hazard.

The spectrograms of the products obtained by the two-step method in Examples 1 and 20 of the present application are shown in FIG. 15 and FIG. 16. The results show that there are fewer impurities, indicating that the two-step method of the present application solves the problems of low product yield and more byproducts in the existing technology. Moreover, the intermediate product of formula III generated in the examples of the present application is not hydrolyzed to generate cyano radicals, thereby greatly improving safety.

### Comparative Example 2 (CN1440376A One-Pot Method)

A mixture of 3-chlorobicyclo[3,2,1]oct-3-en-2-one (12.2 g, 0.078 mol), potassium cyanide (0.25 g, 0.0039 mol, 5 mol %) and methanol (100 mL) was treated with a sodium hydroxide aqueous solution (50%, 21.8 g, 0.273 mol, 3.5 equivalents) and subjected to refluxing for 2 hours. Then the solvent was removed, and the residue was absorbed in diluted hydrochloric acid. The obtained mixture was extracted with ethyl acetate. A n organic phase was dried with sodium sulfate and concentrated.

According to experimental materials and reaction temperatures of CN1440376A, the test was repeatedly carried out many times, and reaction parameters were adjusted many times. The experimental data are shown in Table 7:

TABLE 7
Reaction data for preparing a target product from 3-
chlorobicyclo[3.2.1]-3-octen-2-one by one-pot method

| Serial | | | Cyano | Temperature | Yield of | |
|---|---|---|---|---|---|---|
| number | Solvent | Alkali | reagent | (° C.) | Formula | Note |
| Original | Methanol | Triethylamide | Acetone | 25 | 85% | Original data of |
| record data | | | cyanohydrin | | | Example A of step d |
| | | | | | | of CN1440376A |
| 1 | Methanol | Triethylamide | Acetone | 25 | 80.1% | Data obtained by |
| | | | cyanohydrin | | | repeating |
| 2 | Methanol | Triethylamide | Acetone | 25 | 80.5% | experiment of |
| | | | cyanohydrin | | | CN1440376A by the |
| 3 | Methanol | Triethylamide | Acetone | 25 | 81.2% | inventors |
| | | | cyanohydrin | | | |
| 4 | Methanol | Triethylamide | Acetone | 10 | 77.2% | Data obtained by |
| | | | cyanohydrin | | | optimizing |
| 5 | Methanol | Triethylamide | Acetone | 30 | 81.3% | experiment of |
| | | | cyanohydrin | | | CN1440376A by the |
| 6 | Methanol | Triethylamide | Acetone | 50 | 74.6% | inventors |
| | | | cyanohydrin | | | |

In this comparative example, the intermediate 4-cyanobicyclo[3.2.1]-3-octen-2-one can be generated. Under alkaline conditions, side reactions may occur, which competed with the main reaction, resulting in a decrease in reaction yield.

The gas phase spectrum of the product after extraction, drying and concentration in this comparative example is shown in FIG. 17. The results show that the target product bicyclo[3.2.1]-3-octane-2,4-dione is generated at 12.43 min, which has many impurity peaks and an area normalized purity of 86%. Further purification is required for subsequent reactions. Furthermore, there are many byproducts, so the structure is difficult to separate and identify.

The two-step method of the present application can separate the intermediate 4-alkoxybicyclo[3.2.1]-3-octen-2-one, and the separation process can completely separate the cyanide from the intermediate, reducing the safety risk of acidification to generate hydrogen cyanide. The hydrolysis of the intermediate 4-alkoxybicyclo[3.2.1]-3-octen-2-one has no side reaction competition, simple operation, and high reaction yield. In the one-pot method reaction route of the present application, the intermediate 4-alkoxybicyclo[3.2.1]-3-octen-2-one is generated without separating and purifying the intermediate. The one-pot method reaction route is simple and mild in reaction conditions, high in yield and few in byproducts. In the one-pot method reaction route, a catalytic amount of cyanide is used to ensure safety, and the one-pot method reaction route is suitable for industrial production.

Finally, it should be noted that the above examples are only used to illustrate the technical solutions of the present application and not to limit it; although the present application has been described in detail with reference to the foregoing examples, it will be understood by one of ordinary skill in the art that the technical solutions described in the foregoing examples can still be modified or some technical features can be equivalently substituted; however, these modifications or substitutions do not make the essence of the corresponding technical solutions departing from the spirit and scope of the technical solutions of various examples of the present application.

### INDUSTRIAL APPLICABILITY

The present application discloses a bicyclic ketone compound and a preparation method therefor, and a method for preparing bicyclo[3.2.1]-3-octane-2,4-dione. In the present application, 3-chlorobicyclo[3.2.1]-3-octen-2-one reacts with an alcohol under an anhydrous condition in the presence of a catalyst and a strong alkali to generate 4-alkoxybicyclo[3.2.1]-3-octen-2-one, which is then hydrolyzed to obtain bicyclo[3.2.1]-3-octane-2,4-ditone. According to the present application, the intermediate compound III—4-alkoxybicyclo[3.2.1]-3-octen-2-one is generated through the reaction of 3-chlorobicyclo[3.2.1]-3-octen-2-one with a strong alkali and an alcohol, and then the target product bicyclo[3.2.1]-3-octen-2,4-dione can be obtained in high yield through hydrolysis reaction.

What is claimed is:

1. A bicyclic ketone compound, which is 4-alkoxybicyclo[3.2.1]-3-octen-2-one with a structural formula represented by Formula III: 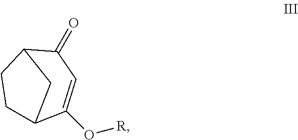
wherein, R is C1-C10 alkyl.

2. The bicyclic ketone compound according to claim 1, wherein R is C1-C5 alkyl, and optionally R is methyl, ethyl, isopropyl, propyl or butyl.

3. A preparation method for the bicyclic ketone compound according to claim 1, comprising following steps: reacting 3-chlorobicyclo[3.2.1]-3-octen-2-one with an alcohol in a presence of a catalyst and an alkaline substance under an anhydrous condition to generate 4-alkoxybicyclo[3.2.1]-3-octen-2-one.

4. A preparation method for the bicyclic ketone compound according to claim 2, comprising following steps: reacting 3-chlorobicyclo[3.2.1]-3-octen-2-one with an alcohol in a presence of a catalyst and an alkaline substance under an anhydrous condition to generate 4-alkoxybicyclo[3.2.1]-3-octen-2-one.

5. The preparation method according to claim 3, wherein the catalyst is one or more selected from the group consisting of cyanides, or the catalyst is one or more selected from the group consisting of potassium cyanide, sodium cyanide, zinc cyanide, nickel cyanide, copper cyanide and acetone cyanohydrin;
and/or, a molar ratio of 3-chlorobicyclo[3.2.1]-3-octen-2-one to the catalyst is 100:1-40, or 100:1-30, or 100:1-20, or 100:1-10, or 100:3-7, or 100:5.

6. The preparation method according to claim 3, wherein the alkaline substance is one or more selected from the group consisting of alkali metal hydroxide, alkali earth metal hydroxide and alcohol alkali; or the alkaline substance is one or more selected from the group consisting of sodium hydroxide, potassium hydroxide, lithium hydroxide, calcium hydroxide, magnesium hydroxide, sodium methoxide, sodium ethoxide, potassium methoxide and potassium ethoxide;
and/or, a molar ratio of 3-chlorobicyclo[3.2.1]-3-octen-2-one to the alkaline substance is 1:1-10, or 1:1-8, or 1:1.5-5, or 1:2-5, or 1:3;
and/or, the alcohol is one or more of methanol, ethanol, propanol, isopropanol, propylene glycol, butanol and pentanol;
and/or, a mass ratio of 3-chlorobicyclo[3.2.1]-3-octen-2-one to the alcohol is 1:1-20, or 1:1-10, or 1:1-5;
and/or, the reaction is carried out in a solvent; optionally, the solvent is a polar non-protonic solvent or a polar protonic solvent; optionally, the solvent is one or more selected from the group consisting of methanol, ethanol, propanol, isopropanol, toluene, xylene, methyl tert-butyl ketone, butyl acetate, dichloromethane and dichloroethane;
and/or, in the reaction of 3-chlorobicyclo[3.2.1]-3-octen-2-one with an alcohol, the reaction temperature is 0° C. to a refluxing temperature of each solvent, or 30° C. to a refluxing temperature of each solvent, or 40° C. to a refluxing temperature of each solvent, or 50° C. to a refluxing temperature of each solvent, or 60° C. to a refluxing temperature of each solvent, or a refluxing temperature of each solvent.

7. A preparation method for bicyclo[3.2.1]-3-octane-2,4-dione, comprising following steps:
reacting 3-chlorobicyclo[3.2.1]-3-octen-2-one with an alcohol in a presence of a catalysts and a strong alkali under an anhydrous condition to generate 4-alkoxybicyclo[3.2.1]-3-octen-2-one, and then conducting hydrolysis to obtain bicyclo[3.2.1]-3-octane-2,4-dione.

8. The preparation method according to claim 7, wherein the catalyst is one or more selected from the group consisting of cyanides, or the catalyst is one or more selected from the group consisting of potassium cyanide, sodium cyanide, zinc cyanide, nickel cyanide, copper cyanide and acetone cyanohydrin;
and/or, a molar ratio of 3-chlorobicyclo[3.2.1]-3-octen-2-one to the catalyst is 100:1-40, or 100:1-30, or 100:1-20, or 100:1-10, or 100:3-7, or 100:5.

9. The preparation method according to claim 7, wherein the strong alkali is one or more selected from the group consisting of alkali metal hydroxide, alkali earth metal hydroxide and alcohol alkali; or the strong alkali is one or more selected from the group consisting of sodium hydroxide, potassium hydroxide, lithium hydroxide, calcium hydroxide, magnesium hydroxide, sodium methoxide, sodium ethoxide, potassium methoxide and potassium ethoxide;
and/or, a molar ratio of 3-chlorobicyclo[3.2.1]-3-octen-2-one to the strong alkali is 1:1-10, or 1:1-8, or 1:1.5-5, or 1:2-5, or 1:3;
and/or, the alcohol is one or more of methanol, ethanol, propanol, isopropanol, propylene glycol, butanol and pentanol;
and/or, a mass ratio of 3-chlorobicyclo[3.2.1]-3-octen-2-one to the alcohol is 1:1-20, or 1:1-10, or 1:1-5;
and/or, the reaction is carried out in a solvent; optionally, the solvent is a polar non-protonic solvent or a polar protonic solvent; optionally, the solvent is one or more selected from the group consisting of methanol, ethanol, propanol, isopropanol, toluene, xylene, methyl tert-butyl ketone, butyl acetate, dichloromethane and dichloroethane;
and/or, in the reaction of 3-chlorobicyclo[3.2.1]-3-octen-2-one with an alcohol, the reaction temperature is 0° C. to a refluxing temperature of each solvent, or 30° C. to a refluxing temperature of each solvent, or 40° C. to a refluxing temperature of each solvent, or 50° C. to a refluxing temperature of each solvent, or 60° C. to a refluxing temperature of each solvent, or a refluxing temperature of each solvent.

10. The preparation method according to claim 7, wherein materials after 3-chlorobicyclo[3.2.1]-3-octen-2-one reacts with an alcohol are treated with any one of methods in following 1) and 2):
1) extraction and liquid separation is performed on the materials after 3-chlorobicyclo[3.2.1]-3-octen-2-one reacts with an alcohol for at least once to obtain an crude oil product, and then conducting hydrolysis; and
2) after 3-chlorobicyclo[3.2.1]-3-octen-2-one reacts with an alcohol, a solvent is removed by evaporation, water is supplemented to continue the reaction, and optionally, after water is supplemented to continue the reaction, acid adjustment is performed to obtain bicyclo[3.2.1]-3-octane-2,4-dione.

11. The preparation method according to claim 7, wherein the hydrolysis is acid hydrolysis or acid adjustment after alkali hydrolysis.

12. The preparation method according to claim 11, wherein the acid used for acid hydrolysis is one or more of hydrochloric acid, sulfuric acid, phosphoric acid, p-methylbenzenesulfonic acid, acetic acid and trifluoroacetic acid;
and/or, a molar ratio of 4-alkoxybicyclo[3.2.1]-3-octen-2-one to the acid is 1:1-10, or 1:1-8, or 1:1-5.

13. The preparation method according to claim 11, wherein the alkali used for alkali hydrolysis is alkali metal hydroxide, alcohol alkali or alkali metal carbonate; or, the alkali used for alkali hydrolysis is one or more of sodium hydroxide, potassium hydroxide, lithium hydroxide, potassium carbonate, sodium carbonate, sodium methoxide and sodium ethoxide;
and/or, a molar ratio of 3-chlorobicyclo[3.2.1]-3-octen-2-one to the alkali is 1:1-10, or 1:1-8, or 1:1.5-5, or 1:2-5, or 1:3.
